(19) United States
(12) Patent Application Publication
(10) Pub. No.: US 2021/0190720 A1
(43) Pub. Date: Jun. 24, 2021

(54) SENSOR AND USES THEREOF IN DETECTING METAL IONS

(71) Applicant: Yissum Research Development Company of the Hebrew University of Jerusalem Ltd, Jerusalem (IL)

(72) Inventors: Shlomo Yitzchaik, Jerusalem (IL); Chaim Gilon, Jerusalem (IL); Mattan Hurevich, Jerusalem (IL); Marx Gerard, Jerusalem (IL)

(21) Appl. No.: 16/640,026

(22) PCT Filed: Aug. 23, 2018
(86) PCT No.: PCT/IL2018/050932

§ 371 Date: Feb. 18, 2020

Related U.S. Application Data
(60) Provisional application No. 62/549,452, filed on Aug. 24, 2017.

(51) Int. Cl. G01N 27/327; G01N 33/84

(52) U.S. Cl. CPC G01N 27/3275; G01N 33/84; G01N 27/3273

(57) ABSTRACT

The invention provides robust, highly sensitive sensors for detecting and determining the presence and quantity of biologically important metal ions in a biological/physiological sample, by utilizing metal binding peptides immobilized on a surface.

The project leading to this application has received funding from the European Union's Horizon 2020 research and innovation programme under grant agreement No 664786.

### TECHNOLOGICAL FIELD

The invention generally pertains to methods of detection.

### BACKGROUND

The human body has an elaborate system for managing and regulating the amount of key trace metals circulating in blood and stored in cells; zinc and copper being essential metal-ions for numerous biochemical processes in the body. Their levels are tightly maintained in all body organs. Impairment of Cu to Zn ratio in serum was found to correlate with many disease states, including immunological and inflammatory disorders, autism, Alzheimer's disease, skin diseases and also cancer.

One of the most common trace-metal imbalances is elevated copper and depressed zinc. Particularly in humans, impaired levels of zinc leads to chronic metabolic disturbances such as atrophy or growth retardation. Quantification of zinc in red blood cells is used to differentiate between Grave's disease and thyrotoxicosis.

Many analytical methods, such as atomic absorption spectroscopy (AA), inductively coupled plasma mass spectroscopy (ICP-MS), inductively coupled plasma atomic emission spectroscopy (ICP-AE) and physicochemical techniques are in use for the detection of Zn and Cu metal ions. Although these methods provide low detection limit and high specificity, the majority of such conventional analytical methods rely on sophisticated, expensive instrumentation and also require tedious sample pretreatment methods and/or operating procedures. These limitations underscore the need for portable (point-of-care) devices, so that the testing can be done conveniently at the time and place of patient care or for field studies.

Electrochemical sensors play a significant role in diagnostic detection of various metabolites in bio-fluids, some of which utilizing biological components such as DNA, enzymes, proteins and peptides as selective recognition elements. Several such sensors have been exploited for the detection of metal-ions. The selective metal ligation of proteins is derived from the specific amino-acids sequence and conformations. Peptides are attractive candidates for the development of ion selective biosensors due to their high sensitivity and specificity. A large variety of strategies such as self-assembled peptides based electrochemical sensors, peptide nano-fibrils, potentiometric stripping analysis at bismuth-film electrode and peptides anchored to aryldiazonium salt grafted graphite electrodes have been reported for metal-ion sensing.

Fogg et al. [1] reported voltammetric determination of Cu²⁺ concentration by pre-formed poly-L-histidine film at a hanging mercury drop electrode.

Chow and Goading [2] showed that while the tripeptide Gly-Gly-His selectively interacts with Cu²⁺, its isomer, Gly-His-Gly, cross reacts with Cu²⁺ and Zn⁺.

Oxytocin (OT) is a metal binding peptide that has an affinity for metal ions and is a highly conserved mediator of physiologic and psychic processes. OT-metal complex interacts with the OT receptor (OTR), which belongs to the G-protein coupled receptor family, in a process that activates several different second messenger systems [3,4]. Binding of OT to different divalent metal, notably with Zn²⁺ or Cu²⁺, affect its interaction with OTR which regulates signaling pathways [5,6].

### BACKGROUND ART

[1] Moreira, J. C.; Zhao, R.; Fogg, A. G. Analyst 1990, 115, 1561-1564.
[2] Chow, E.; Goading, J. J. Electroanalysis 2006, 18, 1437-1448.
[3] Marx, G.; Gilon, C. ACS Chem. Neurosci. 2013, 4, 983-993.
[4] Derek B. Hope, V. V. S. Murti and Vincent du Vigneaud A Highly Potent Analogue of Oxytocin, Desamino-oxytocin J Biol. Chem. 1962, 237:1563-1566.
[5] Jewett, J. C.; Bertozzi, C. R. Chem. Soc. Rev. 2010, 39, 1272-1279.
[6] Zheng, D.; Vashist, S. K.; Dykas, M. M.; Saha, S.; Al-Rubeaan, K.; Lam, E.; Luong, J. H. T.; Sheu, F. S. Materials (Basel). 2013, 6, 1011-1027.

### SUMMARY OF THE INVENTION

It is a purpose of the inventors to provide robust, highly sensitive sensors for detecting and determining the presence and quantity of biologically important metal ions in a biological/physiological sample. As detailed herein, the inventors have developed a methodology for immobilization of metal binding peptides, such as oxytocin (OT) or derivatives thereof, onto a variety of solid surfaces for the purpose of constructing such sensors. The ability of the novel sensors to selectively detect metal ions such as Cu and Zn, in combination, and further in the presence of other metals, using masking agents, or by fine tuning the structure of the metal binding peptides has rendered a highly sensitive and selective sensor device and method for detection of such trace metals. Devices and methods of the invention find their utility not only in the general detection of these critically important metal ions in biological and ecological systems, but more specifically in their ability to determine ion concentration and ratio for the purpose of determining and/or predicting the presence of a certain disease or disorder or the predisposition to suffer from such a disease or disorder.

Provided herein are sensor units, methods for manufacturing the sensor units and methods of detecting a target metal ion with the sensor units.

In a first aspect, a sensor unit is provided that comprises a substrate functionalized with a plurality of metal binding peptides. In some embodiments, each of the plurality of metal binding peptides is associated to the substrate surface or immobilized thereonto directly or indirectly. In some embodiments, the immobilization onto or association with the surface is not via covalent or electrostatic interactions.

The peptides may be mobilized onto a substrate surface region or associated to the substrate surface by any one or more of the following modes of association:

1) indirectly via a linker moiety that is covalently bonded to the metal binding peptide (FIG. 1, FIG. 5, FIG. 15, FIG. 18). As demonstrated herein, the linker moiety may be a mercapto alkanoic acid, wherein the acid functionality permits covalent association with, e.g., an amine group on the metal binding peptide and the mercapto group permits surface association, e.g., to a gold surface (FIG. 18), or the linker moiety may be constructed bottom-up to yield a linker of tailored length, composition, functionalities, etc. (FIG. 1, FIG. 5, FIG. 15);

2) directly via an atom or a group of atoms that is/are native (part of) the metal binding peptide (FIG. 12). As demonstrated herein this may be achieved via dissociation of the ring disulfide bond and subsequent association of the sulfur atoms with a gold surface;

3) via insertion or intercalation in a membrane-like monolayer formed on the surface (FIG. 22). As demonstrated herein, the membrane-like monolayer is a monolayer of surface-associated aliphatic chains, forming a dense layer. The metal-binding peptide is adapted or functionalized with an aliphatic tail capable of intercalating between the surface-associated aliphatic chains. The aliphatic tail of the metal-binding peptide does not associate to the surface, rather undergoes interaction with the exposed aliphatic chains of the monolayer.

The invention further provides a sensor unit comprising a substrate having a surface, a monolayer comprising a plurality of metal binding peptides associated directly or indirectly to the surface, as defined herein, the metal binding peptides being selected to selectively bond or ligate or associate with at least one metal ion. In some embodiments, each of the plurality of metal binding peptides is associated to the substrate surface via a linker moiety that aligns the metal binding peptides perpendicular to the substrate surface. In other words, the peptide used in accordance with the invention is not provided parallel or flat on the substrate surface.

The invention additionally provides a metal binding peptide-based sensor for detecting the presence and determining the amount of at least one metal ion in an aqueous medium, the sensor comprising a plurality of surface-associated metal binding peptide molecules capable of selectively associating to the at least one metal ion.

The metal binding peptide utilized in accordance with the invention is a peptide comprising between 3 and 20 amino acids. In some embodiments, the peptide comprises between 3 and 19, 3 and 18, 3 and 17, 3 and 16, 3 and 15, 3 and 14, 3 and 13, 3 and 12, 3 and 11, 3 and 10, 3 and 9, 3 and 8, 3 and 7, 3 and 6, 3 and 5, 5 and 20, 5 and 19, 5 and 18, 5 and 17, 5 and 16, 5 and 15, 5 and 14, 5 and 13, 5 and 12, 5 and 11, 5 and 10, 5 and 9, 5 and 8, 5 and 7, 10 and 20, 10 and 19, 10 and 18, 10 and 17, 10 and 16, 10 and 15, 10 and 14, 10 and 13, 10 and 12, 15 and 20, 15 and 19, 15 and 18 or between 15 and 17 amino acids. In some embodiments, the peptide comprises 3, 4, 5, 6, 7, 8, 9, 10, 11, 12, 13, 14, 15, 16, 17, 18, 19 or 20 amino acids.

In some embodiments, the metal binding peptide is a cyclic peptide. In some embodiments, the peptide comprises a disulfide bond.

In some embodiments, the metal binding peptide is selected from cyclic and noncyclic metal binding peptides. In some embodiments, the metal binding peptide is selected from oxytocin (OT), somatostatin and vasopressin, and derivatives thereof.

In some embodiments, the metal binding peptide is somatostatin:

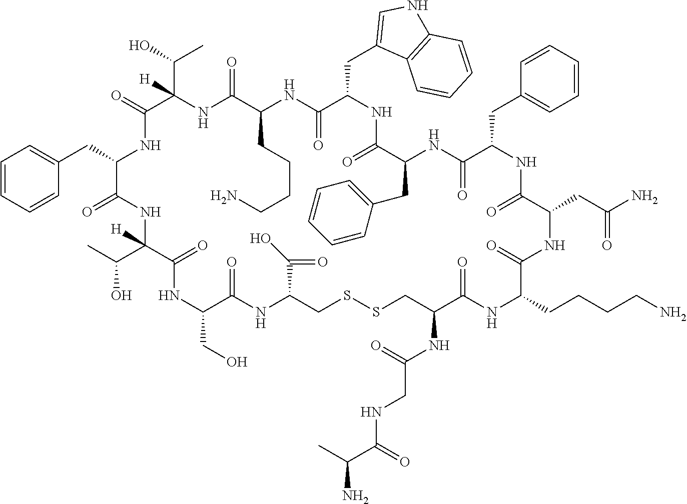 [SMILES: C[C@H](N)C(=O)NCC(=O)N[C@H]1CSSC[C@@H](C(=O)O)NC(=O)[C@H](CO)NC(=O)[C@H]([C@@H](C)O)NC(=O)[C@H](Cc2ccccc2)NC(=O)[C@H]([C@@H](C)O)NC(=O)[C@H](CCCCN)NC(=O)[C@H](Cc2c[nH]c3ccccc23)NC(=O)[C@H](Cc2ccccc2)NC(=O)[C@H](Cc2ccccc2)NC(=O)[C@H](CC(N)=O)NC(=O)[C@H](CCCCN)NC1=O]

In some embodiments, the metal binding peptide is a somatostatin derivative, wherein one or more of the amine moieties of somatostatin is derivatized, substituted or otherwise modified with a functional group. In some embodiments, the amine moieties are alkylated or functionalized. In some embodiments, wherein the amine groups are amide groups, the amide groups are alkylated. In some embodiments, the alkylating moiety is a short alkyl group comprising between 1 and 5 carbon atoms. In some embodiments, the alkyl is selected from methyl, ethyl, propyl, butyl and pentyl. In some embodiments, the alkyl is methyl or ethyl or propyl or butyl or pentyl. In some embodiments, the alkyl is methyl or ethyl.

In some embodiments, where the amine groups are not amide groups, rather selected from —NH— and —NH₂ groups, they may be alkylated or functionalized by, e.g., acylation with a fatty acid or an organic acid group.

In some embodiments, any of amine moieties may act as point of connectivity to the surface or to a linker moiety. In some embodiments, the metal binding peptide is vasopressin:

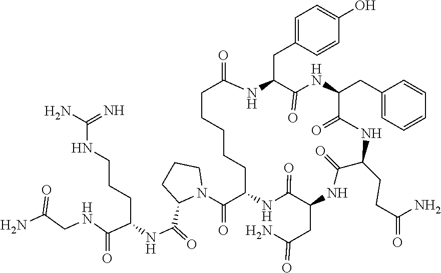 [SMILES: N=C(N)NCCC[C@H](NC(=O)[C@@H]1CCCN1C(=O)[C@@H]1CCCCCC(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N1)C(=O)NCC(N)=O]

In some embodiments, the metal binding peptide is a vasopressin derivative, wherein one or more of the amine or amide moieties of vasopressin is derivatized, substituted or otherwise modified with a functional group. In some embodiments, the amine or amide moieties are alkylated or functionalized. In some embodiments, wherein the amine groups are amide groups, the amide groups are alkylated. In some embodiments, the alkylating moiety is a short alkyl group comprising between 1 and 5 carbon atoms. In some embodiments, the alkyl is selected from methyl, ethyl, propyl, butyl and pentyl. In some embodiments, the alkyl is methyl or ethyl or propyl or butyl or pentyl. In some embodiments, the alkyl is methyl or ethyl.

In some embodiments, where the amine groups are not amide groups, rather selected from —NH— and —NH₂ groups, they may be alkylated or functionalized by, e.g., acylation with a fatty acid or an organic acid group.

In some embodiments, any of amine moieties may act as point of connectivity to the surface via any of the modes recited hereinabove.

In some embodiments, the metal binding pentide is oxvtocin:

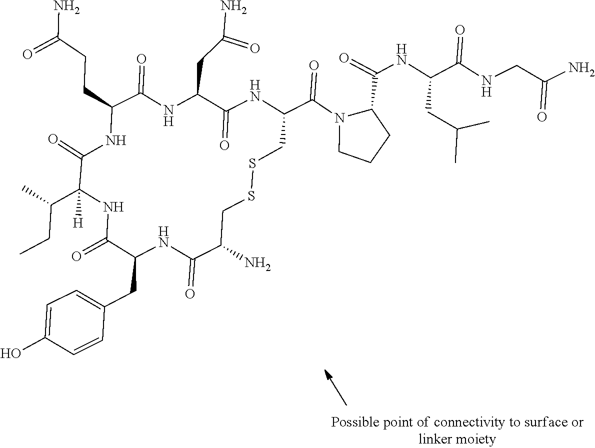 [SMILES: CC[C@H](C)[C@@H]1NC(=O)[C@H](Cc2ccc(O)cc2)NC(=O)[C@@H](N)CSSC[C@@H](C(=O)N2CCC[C@H]2C(=O)N[C@@H](CC(C)C)C(=O)NCC(N)=O)NC(=O)[C@H](CC(N)=O)NC(=O)[C@H](CCC(N)=O)NC1=O]

In some embodiments, the metal binding peptide is N-alkylated oxytocin. In some embodiments, the N-alkylation may be at any N atom of the oxytocin molecule. In some embodiments, the alkylating moiety is a short alkyl group comprising between 1 and 5 carbon atoms. In some embodiments, the alkyl is selected from methyl, ethyl, propyl, butyl and pentyl. In some embodiments, the alkyl is methyl or ethyl or propyl or butyl or pentyl. In some embodiments, the alkyl is methyl or ethyl.

In some embodiments, the metal binding peptide is N-methyl oxytocin. In some embodiments, the N-methyl oxytocin is of the following structure:

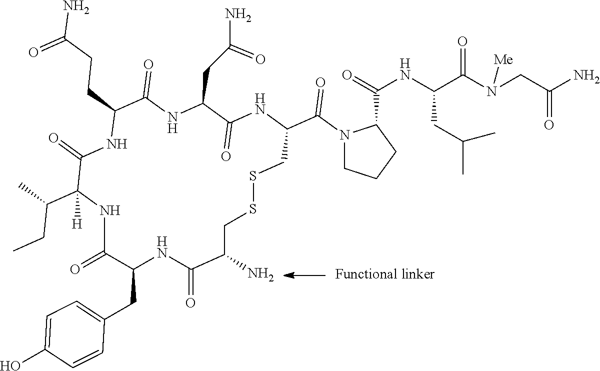 [SMILES: CC[C@H](C)[C@@H]1NC(=O)[C@H](Cc2ccc(O)cc2)NC(=O)[C@@H](N)CSSC[C@@H](C(=O)N2CCC[C@H]2C(=O)N[C@@H](CC(C)C)C(=O)N(C)CC(N)=O)NC(=O)[C@H](CC(N)=O)NC(=O)[C@H](CCC(N)=O)NC1=O]

In some embodiments, the metal binding peptide is of the general formula I:

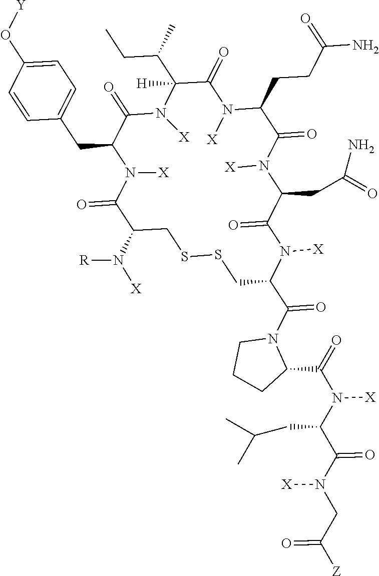

wherein

X is H or a C₁-C₁₆ alkyl;

R is H or a functional group permitting association to the surface or to a bifunctional moiety, as disclosed herein;

Y is selected from H, PO₃⁻², SO₃⁻¹ and glycan.

In some embodiments, each of X is H.

In some embodiments, each of X is an alkyl selected from methyl, ethyl, propyl, butyl and pentyl. In some embodiments, each X is methyl.

In some embodiments, Y is H.

In some embodiments, Y is a glycan. In some embodiments, the glycan is selected amongst natural and synthetic carbohydrates. Non-limiting examples of glycans include glucose, galactose, mannose and their C-2 deoxy analogs, their C-6 deoxy analogs, mucin antigens, silylated glycans, arabinogalactans, polymannans, poly-glucose, poly N-acetyl glucose and others.

In some embodiments, R is a C₅-C₁₅ alkyl group, a —(C═O)C₅-C₁₅ alkyl group, —O—(C═O)C₅-C₁₅ alkyl group, a C₅-C₁₅ alkyl-S— group, a —(C═O)C₅-C₁₅ alkyl-S— group, —O—(C═O)C₅-C₁₅ alkyl-S— group, an amine group, an amide, a carboxylic acid, an aldehyde, a ketone, an alcohol, a halide, an acyl, aryl moieties, activated aryl moieties (such as benzyne) and an azide group.

In some embodiments, R is a C₅-C₁₅ alkyl group, a —(C═O)C₅-C₁₅ alkyl group, —O—(C═O)C₅-C₁₅ alkyl group, a C₅-C₁₅ alkyl-S— group, a —(C═O)C₅-C₁₅ alkyl-S— group or a —O—(C═O)C₅-C₁₅ alkyl-S— group, wherein in each of the groups containing a sulfur atom, the sulfur is an atom associating to a surface of the substrate (may be presented in a form such as —SH).

In some embodiments, R is an azide group. In some embodiments, the azide groups has the structure —[C(═O)]ₙ—C₁-C₁₀alkylene-N₃, wherein n is zero or 1. In some embodiments, R is selected from —C(═O)—C₁-C₁₀alkylene-N₃ and —C₁-C₁₀alkylene-N₃, wherein the C₁-C₁₀alkylene is selected from methylene, ethylene, propylene, butylene, pentylene, hexylene, heptylene, octylene, nonylene and decylene, wherein each of the C₁-C₁₀ alkylene is optionally substituted with a thiol group.

In some embodiments, the C₁-C₁₀alkylene is methylene. In some embodiments, R is —C(═O)—CH₃—N₃ and —CH₃—N₃.

As noted hereinabove, the metal binding peptides may be directly associated to a surface region of the substrate or indirectly via a linker moiety that is bifunctional, having at least one moiety or group that is capable of associating to the surface and at least one moiety or group that is capable of associating to the metal binding peptides at a point of connectivity (an atom or a group of atoms on the metal binding peptides that permits chemical association with the bifunctional molecule). Thus, where surface association via a linker moiety is desired, the metal binding peptides may be chemically modified to contain one or more active groups that permit association with the linker moiety. Such groups may be selected from an alkyl group, a fatty acid group, an alkyl thiol group, an amine group, an amide, a carboxylic acid, an aldehyde, a ketone, an alcohol, an halide, an acyl, benzyne moieties, a moiety comprising one or more double bonds, a moiety comprising one or more triple bonds, activated aryl moieties and an azide. Depending on the selected group on the metal binding peptides, modification thereof may be carried according to known procedures. A person of skill in the art would know to select a specific point of connectivity on the metal binding peptides and chemically modify it to permit, improve or otherwise control association with the linker moiety.

Similarly, the association with the linker moiety may be selected to proceed in any one way, as known in the art, to afford covalent bonding between the metal binding peptides and the linker moiety. For example, chemical association may be achieved by one or more of addition reactions, elimination reactions, radical reactions, substitution reactions, redox reactions, rearrangement reactions, polymerization reactions, cycloaddition reactions, and others.

Thus, the invention further provides a method for fabricating a sensor unit according to the invention, the method comprising forming on a surface region of a substrate an active monolayer comprising a plurality of metal binding peptide molecules, said metal binding peptide molecules being associated with said surface region through a mode of association as disclosed herein.

The fabrication method may comprise a plurality of steps permitting bottom-up construction of the active monolayer or a single step involving direct deposition of the metal binding peptides (with or without a linker moiety) onto the surface region.

In a bottom-up construction, the method comprises surface-associating a plurality of bifunctional molecules (linkers), each having at least one surface-associating moiety and at least one moiety engineered or selected to permit chemical association with the plurality of metal binding peptide molecules, e.g., such that each of the bifunctional molecules is associated to the surface and to one or more metal binding peptides. In some embodiments, each of the bifunctional molecules is capable of associating to a single metal binding peptide molecule.

In some embodiments, the bifunctional molecules may be constructed on the surface from preselected building blocks, thereby controlling the length and thus the distance of the metal binding peptides from the surface. In other words, the final length of the linkers may be determined and achieved by step-wise extension of a first deposited group. An exemplary bottom-up construction is depicted in Scheme 1 below:

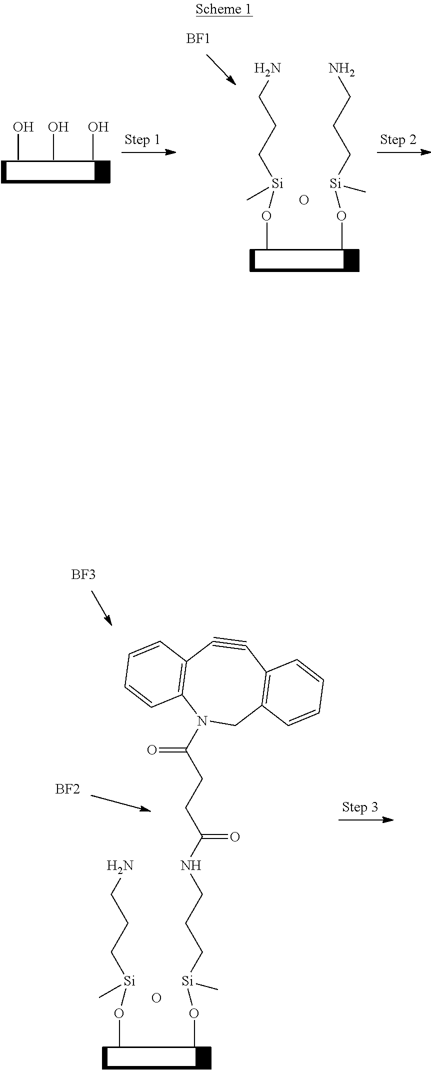

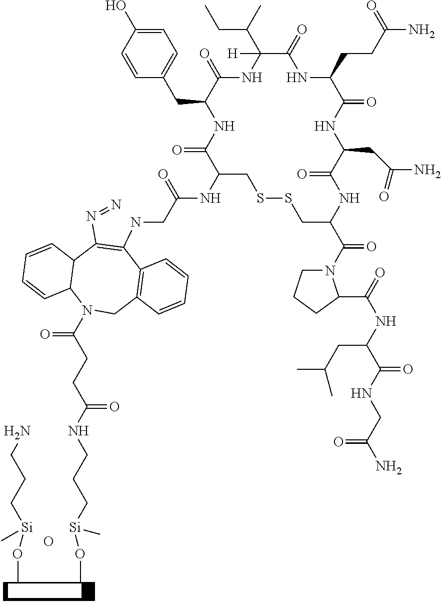

As depicted in Scheme 1 for the purpose of exemplifying the bottom-up construction of a sensor unit according to the invention, in step 1 of a sequence of linker extensions, a first bifunctional material (BF1) is deposited on a surface region. The first bifunctional material (BF1) has 3 carbon atoms, one surface associating group (Si) and one group through which chain extension is made possible (—NH₂). After a first layer of BF1 is formed, in step 2 the layer is reacted with a second bifunctional material (BF2) having a desired number of carbon atoms, one functional group (in the exemplified case an acyl or an activated carbonyl) to associate with the exposed functional group (—NH₂) in layer BF1, and one functional group (a carboxyl or an activated carbonyl) that is selected to either associate to a further bifunctional material (BF3) or to the metal binding peptide. In the example shown in Scheme 1 chain extension of BF1 is with a linker moiety that comprises both BF2 and BF3. The metal binding peptide is subsequently associated to the end group in BF3.

Similarly, the three block linker, comprising BF1, BF2 and BF3 may be formed in advance and deposited as one linker on the surface region. The metal binding peptide may also be associated with the linker prior to surface deposition.

In some embodiments, the method comprises surface-associating a plurality of bifunctional molecules of a first length (chain length: number of atoms, number of functionalities, etc) and chain-extending said bifunctional molecules of a first length to afford a plurality of bifunctional molecules of a second length (being different from the first length). In some embodiments, the bifunctional molecules of the first length have a terminus group selected to permit chain extension; such terminus group may be selected from an amine group, an amide, a carboxylic acid, an aldehyde, a ketone, an alcohol, an halide, an acyl, and others.

In some embodiments, the chain extended bifunctional molecules, having a second length, or the final linker moieties, have a terminus group selected to permit further chain extension or chemical association with the metal binding peptide. Where further chain extension is desired, the terminus groups may be selected as above. Where chemical association with the metal binding peptides is desired, the terminus groups may be selected from an amine group, an amide, a carboxylic acid, an aldehyde, a ketone, an alcohol, an halide, an acyl, benzyne moieties, a moiety comprising one or more double bonds, a moiety comprising one or more triple bonds, activated aryl moieties and an azide.

In some embodiments, the linker has a metal binding peptide-associating moiety that is reactive towards at least one functional group on the metal binding peptide molecule, to permit covalent association as explained herein. For example, where the functional group on the metal binding peptide is an electrophile, the linker may comprise a nucleophilic group, and vice versa. Similarly, where the functional group on the metal binding peptide is an azide, the linker may comprise a terminal or internal alkyne to permit 1,3-dipolar cycloaddition between the azide and the alkyne, and vice versa.

As used herein, the “linker moiety” or molecule through which the metal binding peptide is associated to the surface is a bifunctional molecule having at least one surface associating group and at least one group capable of associating to the metal binding peptide. This bifunctional molecule may be surface constructed from short moieties, as described herein, or may be prepared in advance as such. The linker molecule is typically a linear atom chain, e.g., carbon chain, comprising between 2 and 20 atoms, e.g., carbon atoms. In some embodiments, the atom chain may comprise between 2 and 20 carbon atoms and one or more inner-chain groups selected from heteroatoms (N, O, S), amine groups (—NH—, ═N—, —N(R)—, wherein R is an amine substituting group), carbonyl groups (—C(═O)—, —C(═O)—O—, —C(═O)—NR—, —O—C(═O)—, —NR—C(═O)—, —NR—C(═O)—NR—, wherein R is an amine substituting group), arylene (e.g., phenylene, naphthylene), carbocyclyl (cyclopropylene, cyclopentylene, cyclohexylene) and cyanuric acid.

In some embodiments, the bifunctional molecules (linkers) are selected amongst amide-containing carbon chains (e.g., —C₁-C₂₀ alkylene-C(═O)—NR—C₁-C₂₀ alkylene- and —C₁-C₂₀ alkylene-NR—C(═O)—C₁-C₂₀ alkylene-, provided that the total number of carbon atoms does not exceed 20, and wherein R is a nitrogen substituting group), urea-containing carbon chains (e.g., —C₁-C₂₀alkylene-NR—C(═O)—NR—C₁-C₂₀ alkylene-, provided that the total number of carbon atoms does not exceed 20, wherein R is a nitrogen substituting group), imide-containing carbon chains (e.g., —C₁-C₂₀ alkylene-C(═O)—NR—C(═O)—C₁-C₂₀ alkylene-, provided that the total number of carbon atoms does not exceed 20; wherein R is a nitrogen substituting group), ester-containing carbon chains (e.g., —C₁-C₂₀ alkylene-C(═O)—O—C₁-C₂₀ alkylene- and —C₁-C₂₀ alkylene-O—C(═O)—C₁-C₂₀ alkylene-, provided that the total number of carbon atoms does not exceed 20), anhydride-containing carbon chains (e.g., —C₁-C₂₀alkylene-C(═O)—O—C(═O)—C₁-C₂₀ alkylene-, provided that the total number of carbon atoms does not exceed 20), ketones (e.g., —C₁-C₂₀ alkylene-C(═O)—C₁-C₂₀ alkylene-, provided that the total number of carbon atoms does not exceed 20), ethers (e.g., —C₁-C₂₀alkylene-O—C₁-C₂₀ alkylene-, provided that the total number of carbon atoms does not exceed 20), dialkyl or trialkyl amines (e.g., —C₁-C₂₀alkylene-NR—C₁-C₂₀alkylene-, provided that the total number of carbon atoms does not exceed 20; wherein R═H or an alkyl), carbamates, ethers and flouroalkanes.

In some embodiments, the linker moiety is constructed of a linear chain comprising between 4 and 20 carbon atoms, the chain interrupted by one or more atoms selected from N, O and S and groups selected from —C(═O)—NR—, —NR—C(═O)—, —NR—C(═O)—NR—, —C(═O)—NR—C(═O)—, —C(═O)—O—, —O—C(═O)—, —C(═O)—O—C(═O)—, —C(═O)—, wherein R═H or a C₁-C₅alkyl or alkylene.

In a bifunctional molecule utilized according to the invention, the surface associating moiety and the cyanuric acid associating moiety are different. The surface associating moiety may be selected depending, inter alia, on the surface (e.g., material composition and physical characteristics) to which association is desired and the type of association desired. The surface associating moiety may be selected also based on surface functionalities that may or may not be present (e.g., existing functional groups with which chemical association may be achieved). In some embodiments, the surface associating groups may be selected from —OH, —SH, —S—S—, —SeH, —Se—Se—, Si, —SiO₂, chlorosilanes, alkoxysilanes, carboxyl groups, amine groups, acyl groups, acyl-x (wherein x may be selected from halides, cyanides, azides, succinimide) maleimide, azide, alkynes, epoxides, phosphonates and others. In some embodiments, where the surface to be associated to is gold, the peptide may comprise a surface associating groups such as —SH or —S—S—. In some embodiments, the peptide comprises a disulfide group —S—S— enabling surface association via disulfide dissociation (exemplifying direct surface association as disclosed herein).

In some embodiments, the association between the metal associating peptide and the surface is via insertion or intercalation of a fatty acid tail present on the metal associating peptide into a membrane-like film or monolayer of hydrophobic molecules. The membrane-like film or layer is formed of straight alkyl thiols of a length of between 10 and 30 carbon atoms.

The surface to which association is required may be a surface region of any solid substrate. The surface material may be the same as the substrate material, or may be of a different material (composition). For example, the substrate may be of one material, while the surface thereof may be an oxide of that material. Similarly, the substrate may be of one material and the surface may be a film of a different material, the film may be native to the substrate material or may be fabricated on top of the surface. In some embodiments, the surface material is selected from oxides (of transition metals including lanthanides and actinides), glass, metal such as gold, carbon allotropes and glassy carbon.

In some embodiments, the surface and the substrate materials are the same.

In some embodiments, the surface is a surface region of an electrode or an electrode assembly.

The surface onto which the sensing molecules (linker and metal binding peptide) are deposited or to which are associated, need not be fully covered with the sensing molecules. The density of the sensing molecules on the surface may vary. For example, an active monolayer may be formed of surface associated linker molecules which at least 10% of which are further associated with metal binding peptide moieties. In some embodiments, at least 10, 20, 30, 40, 50, 0, 70, 80, or 90% of the surface associated linker molecules are further associated with metal binding peptide moieties.

In some embodiments, an active monolayer may comprise a homogenous distribution of linker moieties associated with metal binding peptide moieties and linker moieties that are not associated with metal binding peptide moieties. The ratio between those which are associated and those that are not associated with metal binding peptide moieties may be between 0.01:1 and 1:0.01. In some embodiments, the ratio is 1:1.

The invention further provides a method comprising contacting a sensor unit according to the invention with a sample that comprises or that is suspected of comprising at least one metal ion, and determining one or both of presence and amount of said at least one metal ion in said sample. In some embodiments, the method further comprises measuring a relative ratio between two or more metal ions present in the sample.

The invention further provides a method for detecting a target metal ion with a sensor unit, the method comprising providing a sensor unit having surface-associated metal binding peptide molecules; permitting association of metal ions to the metal binding peptide molecules; and measuring at least one signal indicative of the presence and quantity of the metal ions.

The invention further provides a method for determining the presence of a target metal ion in a sample, the method comprising providing a sensor unit having surface-associated metal binding peptide molecules; permitting association of metal ions to the metal binding peptide molecules; and measuring at least one signal indicative of the presence of the metal ions in the sample.

The invention further provides a method for quantifying a target metal ion in a sample, the method comprising providing a sensor unit having surface-associated metal binding peptide molecules; permitting association of metal ions to the metal binding peptide molecules; and measuring at least one signal indicative of the amount of the metal ions in the sample.

The metal ions that may be detected and quantified according to methods of the invention are zinc and copper. A person of skill would appreciate that the mechanism by which the metal ions are bonded or associated to the metal binding peptides may vary and has no bearing on the invention. Without wishing to be bound by a specific mode of action of mechanism, it is believed that the metal binding peptides utilized according to the invention have increased affinities towards the selected metal ions. The increased affinity towards the zinc and copper ions render it possible to detect their presence and quantities in any aqueous medium, whether physiological or non-physiological. Such samples may be tested for the presence and quantity of these metals for general diagnostic or evaluation purposes, for medicinal purposes or for any other purpose. Physiological samples may be blood, plasma serum, urine, saliva and CSF (cerebrospinal fluid).

Methods of the invention can detect the presence of as little as 100 fM of Zn and as little as 500 fM of Cu.

The invention further provides a method of diagnosing the existence of at least one disease or disorder or predicting the occurrence of said disease or disorder or determining the prevalence of the disease or disorder in a subject or subject population, the disease or disorder being characterized by a chronic or acute abnormality in zinc and/or copper levels (which may be deficient or excessive) in the subject, the method comprising using a sensor unit or device according to the invention, in a sample obtained from the subject, to determine one or more of zinc level, copper level and/or the ratio between the levels of zinc and copper in the sample; and comparing said zinc level, copper level and/or ratio of levels to a normal level thereof; wherein a deviation from said normal level being indicative of (or a tool in determining) the presence, prevalence or occurrence of the disease or disorder.

In some embodiments, the disease or disorder characterized by an impairment in the levels of zinc and/or copper is selected from immunological and inflammatory disorders, autism, Alzheimer's disease, multiple sclerosis, skin diseases, Grave's disease, thyrotoxicosis and cancer.

The selectivity of methods of the invention is reflected not only in the ability to selectively measure zinc and copper concentrations when in the presence of other metal ions, which may or may not be present in greater amounts, but also in the ability to distinguish between zinc and copper. The selectivity may be achieved by engineering the structure of the metal binding peptides to have a greater affinity towards one of the two metal ions (e.g., by changing the linker moiety or the mode of association as disclosed herein), or by measuring their presence or concentration in the presence of a metal masking agent, or by changing sample environment such as pH, ionic strength, counter ion, etc. For instance, when a method of the invention is used for selective detection of copper ions, a zinc masking agent would be used. Similarly, when a method of the invention is used for selective detection of zinc ions, a copper masking agent may be used. The masking agent is a material capable of interacting (complexing) with the metal ion, rendering it unavailable for detection by methods of the invention.

In some embodiments, the copper masking agent is a material capable of forming a complex with copper, but not with zinc. The copper masking material may be selected from thiourea, 2,3-dimercaprol, 8-hydroxyqunoline, meso-2,3-dimercaptosuccinic acid, triethylenetetramine (TETA), Trientine (TETA dihydrochloride) and others.

In some embodiments, the zinc masking agent is a material capable of forming a complex with zinc, but not with copper. The zinc masking material may be selected from pyrophosphate, N,N,N′,N″-tetrakis(2-pyridylmethyl)-ethylenediamine (TPEN), calcium ethylenediaminetetraaceticacid (CaEDTA), (4-[2-(bis-pyridin-2-ylmethyl amino)ethylamino]-methylphenyl)methanesulfonic acid, sodium salt (DPESA), and 4-([2-(bis-pyridin-2-ylmethylamino)ethyl]pyridin-2-ylmethylamino-methyl)phenyl] methanesulfonic acid, sodium salt (TPESA). Alternatively, the presence of zinc may be masked at high pH, e.g, to thereby enhance affinity towards copper ions while dramatically reducing sensor sensitivity towards zinc ions.

The association of the metal ions with the metal binding peptides at the surface of the active monolayer can be detected in a variety of detection methods including, but not limited to, optical detection (where spectral changes occur upon changes in redox states) such as fluorescence, phosphorescence, luminescence, chemiluminescence, electrochemiluminescence and refractive index; and electronic detection such as amperommetry, voltammetry, capacitance and impedance. These methods include time or frequency dependent methods based on AC or DC currents, pulsed methods, lock-in techniques, filtering and time-resolved techniques.

In some embodiments, the active layer comprising the metal binding peptide, as defined herein, may be directly deposited or fabricated on a surface region of an electrode. Thus, a sensor device of the present invention includes an electrode capable of specifically sensing a metal ion to be detected. The metal ion may be sensed directly through electro-oxidation on a metallic electrode or through sensing elements which are in electrical contact with the electrode.

Thus, the invention further provides an electrode and an electrode assembly.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 provides a general scheme of OT-Sensor/OT-Wafer step wise preparation, Step 1) APTES modification on GCE/Si-Wafer active hydroxyl, Step 2) DBCO-NHS coupling to the amine on the interface. Step 3) Azido-OT coupling to DBCO by click chemistry.

FIGS. 2A-D present atomic force microscopic images (area: 1.0 μm×1.0 μm) recorded for OT immobilized Si disc (OT-Wafer) of: (FIG. 2A) hydroxylated silicon wafer (ρ=2.0 Å) (FIG. 2B) APTES modified silicon wafer (ρ=2.2 Å) (FIG. 2C) DBCO modified silicon wafer (ρ=2.5 Å) and (FIG. 2D) OT-Wafer (ρ=2.9 Å).

FIGS. 3A-B present XPS spectra of OT-Wafer before (line a in FIG. 3A and line c in FIG. 3B) and after incubation in (line b in FIG. 3A) 1 μM Zn²⁺ and (line d in FIG. 3B) 1 μM Cu²⁺ solution.

FIG. 4 provides Nyquist plots obtained for the various assembly steps on the GC electrodes; (a) bare GCE, (b) GCE-NH₂, (c) GCE-DBCO, (d) OT-Sensor and (e) OT-Sensor incubated in 1 nM Zn²⁺ solution (electrolyte: 5 mM [Fe(CN)₆]^{3-/4-} consists of 0.1 M PBS at pH 7.0).

FIG. 5 is a schematic showcase of redox couple diffusion pathway on modified electrode through the organic layer to the GCE surface. There are 2 diffusion pathways: one through the OT-Ring and the other through the OT-Tail.

FIGS. 6A-B provide: (FIG. 6A) Nyquist plots obtained for OT-Sensor in 5 mM [Fe(CN)₆]^{3-/4-} consists of 0.1 M PBS at pH 7.0 after incubation in various Zn²⁺ concentrations; (a) blank solution (b) 10⁻¹² M Zn²⁺ (c) 10⁻¹¹ M Zn²⁺ (d) 10⁻¹⁰ M Zn²⁺ (e) 10⁻⁹ M Zn²⁺ (f) 10⁻⁸ M Zn²⁺ and (g) 10⁻⁷ M Zn²⁺ (inset: enlarged Nyquist plots); and (FIG. 6B) logarithmic concentration of Zn²⁺ vs. normalized charge transfer resistance (R_{CT}) of OT-Ring (SR), OT-Tail (ST) and solution resistance (Rₛ) with a slope of 0.10 (R_{SR}), 0.11 (R_{ST}) and 0.005 dec⁻¹ (Rₛ).

FIGS. 7A-B provide: (FIG. 7A) Nyquist plots obtained for OT-Sensor in 5 mM [Fe(CN)₆]^{3-/4-} consists of 0.1 M PBS at pH 7.0 after incubation in various Cu²⁺ concentrations; (a) blank solution (b) 10⁻¹² M Cu²⁺ (c) 10⁻¹¹ M Cu²⁺ (d) 10⁻¹⁰ M Cu²⁺ (e) 10⁻⁹ M Cu²⁺ (f) 10⁻⁸ M Cu²⁺ and (g) 10⁻⁷ M Cu²⁺ (inset: enlarged Nyquist plots); and (FIG. 7B) logarithmic concentration of Cu²⁺ vs. normalized charge transfer resistance (R_{CT}) of OT-Ring (SR), OT-Tail (ST) and solution resistance R_{CT}(S) with a slope of 0.06 (R_{SR}), 0.16 (R_{ST1}), 0.72 (R_{ST2}) and 0.005 dec⁻¹ (Rₛ).

FIG. 8 shows the response of the OT-Sensor towards various metal ions in 1 nM concentration.

FIG. 9 provides histograms showing simultaneous detection of 1 nM Zn²⁺ and 1 nM Cu²⁺ in a 1:1 mixture in the presence and absence of masking agent 10 M thiourea (TU) and 10 M pyrophosphate (PP).

FIGS. 10A-B provide FIG. 10A: Somatostatin (SSt) assembly on gold electrode: real impedance vs. time was measured at 18° C., 0.1 mM SSt in Tris buffer at pH 7.0; FIG. 10B: Impedimetric response of SSt functionalized electrode to ZnCl titration.

FIG. 11 summarizes a study of the effect of pH on the selectivity of sensors of the invention, as measured using impedimetric measurements of OT-GCE after incubating in 1 nM Zn²⁺ or Cu²⁺ both at pH 7.0 and at pH 10.0.

FIG. 12 demonstrates direct OT assembly on a gold substrate.

FIG. 13 demonstrates dose response of Au-OT sensor (of direct OT assembly) to Cu²⁺ ions and Zn²⁺ ions.

FIG. 14 shows cyclic voltammetry analysis of OT layer (of direct OT assembly) in the presence of Cu²⁺ ions.

FIGS. 15A-F provide the structures of 8-N-Me-OT, 9-N-Me-OT and 8,9-di-N-Me-OT and 2-N-Me-OT, 3-N-Me-OT and 2,3-di-N-Me-OT.

FIG. 16 shows methylated OTs response to zinc in phosphate buffer.

FIG. 17 shows methylated OTs response to copper in phosphate buffer.

FIG. 18 demonstrates fabrication of Au-MOA-OT sensor: shematic representation of a step-wise anchoring of OT on Au surface vio bifunctional thiooctanoic acid linker (MOA): (step A) self-assembling of MOA (step B) coupling of OT to MOA through amide bond and (step C) passivation with 6-mercaptohexanol.

FIGS. 19A-B present metal response of Au-MOA-OT: (FIG. 19A) Nyquist plot showing increase in the R_{CT} of the semi-circle with increase in concentration of Zn²⁺ and (FIG. 19B) corresponding dose response (calibration) curve plotted showing the high sensitivity of sensor towards Zn²⁺ in comparison to Cu²⁺ (▪ Zinc and ▾ Copper).

FIG. 20 demonstrates response of Au-MOA-OT to Cu²⁺: a Nyquist plot showing increase in the R_{CT} of the semi-circle with increase in concentration of Cu²⁺.

FIGS. 21A-B presents: (FIG. 21A) Difference between real impedance (Z′) before and after exposure of Au-MOA-OT to 1 nM Zn²⁺; the peak corresponds to the optimum frequency that impedance signal arises from (FIG. 21B) Time-resolved changes in real impedance (Z) for the of Au-MOA-OT sensor over a time period of 15 minutes at constant frequency 20 Hz. Suitable aliquots of Zn²⁺ are added to the ammonium acetate buffer solution at 580 seconds and 670 seconds.

FIG. 22 depicts hexadecanethiol monolayer (HDT) on gold intercalated with dodecanoic-oxytocin (DOT).

FIG. 23 demonstrates electrochemical Impedance Spectroscopy of HDT modified gold electrode (red), and dodecanoic-oxytocin (DOT) modified gold electrode (blue).

FIG. 24 shows a dose response of Au-HDT-DOT for Zn²⁺ ions.

FIG. 25 demonstrates frequency changes of QCM of DOT adsorption on HDT monolayer.

### DETAILED DESCRIPTION OF EMBODIMENTS

1. Results and Discussion

1.1 Assembly of OT-Sensor and OT-Wafer—The metal binding peptide oxytocin (OT) contains a disulfide bond that is essential for its bioactivity, but the disulfide bond might interact with gold electrode surface and alter the bioactive conformation of OT. Therefore, for the purpose of exploring OT as a potential sensing molecule, OT was deposited on a glassy carbon electrode (GCE) to avoid such unwanted interactions. The OT was deposited on the surface of the GCE via its amino terminus. It is known that the amino terminal group of OT is not essential for OT binding and activation of its receptor since desamino-OT (1-β-mercaptopropionic acid oxytocin) is more potent than OT.

OT was attached to GCE using the non-Cu click chemistry. Click chemistry is very useful to attach unprotected peptides to surfaces since the nucleophilic functional groups on the amino acid residues side chains do not participate in the coupling reaction to the surface. The fabrication process of the OT-Sensor was confirmed by following the physical characterization of OT immobilized on silicon wafer (OT-Wafer) in the same manner as the OT-Sensor

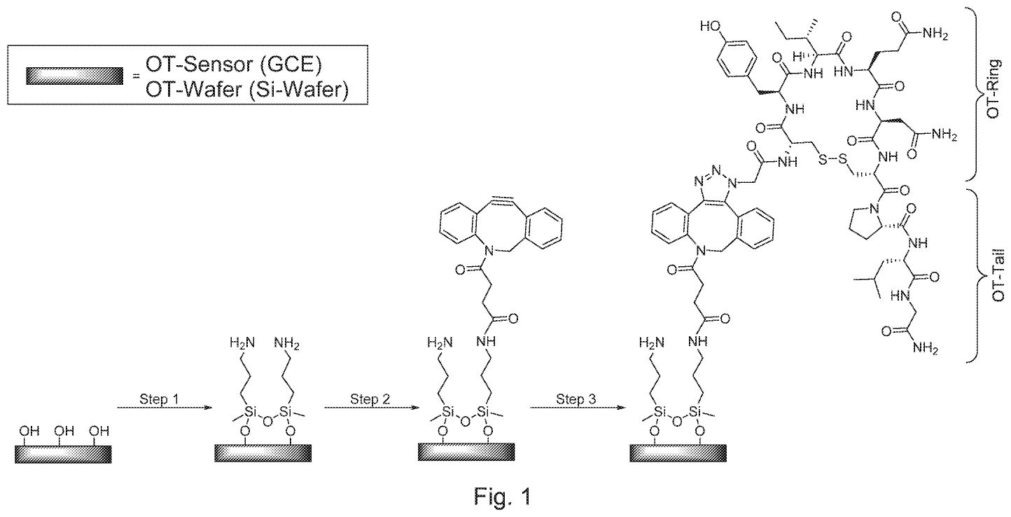

The fabrication of the OT-Sensor and OT-Wafer is shown in FIG. 1. The fabrication was carried out in multiple steps. Initially, hydroxyl functionalization of mirror finished GCE was performed by suspending the electrode in a stirred solution of 1% aqueous solution of KOH. This resulted in GCE surface consists of 94.8% C, 5.2% O, compared to 95.5% C and 4.5% O— obtained for the untreated GCE. Aminopropyl groups on the GCE were generated by reacting the hydroxyl groups of GCE-OH with 3-aminopropyl(triethoxysilane) (APTES) (FIG. 1, step 1). The amino groups were then reacted with dibenzooctyl-N-hydroxy succinimidyl ester (DBCO-NHS) in ethanol (FIG. 1, step 2). The mechanism is similar to the EDC/NHS chemistry for coupling of amino groups and carboxylic acid to form amide bond. The use of DBCO on the surface of GCE enabled the attachment of N(2-azidoacetylyl)-oxytocin (OT-AZ) to the cyclooctyne functionalized GCE via click-chemistry in the absence of copper (FIG. 1, step 3).

1.2 Monolayer characterization The fabrication process of the OT-Sensor was confirmed by following the physical characterization, such as monolayer thickness and surface roughness, of OT immobilized on OT-wafer in the same manner as the OT-Sensor.

1.2.1 Ellipsometry studies of OT-wafer Spectroscopic ellipsometry were a convenient and accurate technique for the measurement of thickness and optical constants based on the changes in the state of polarization light upon reflection of light from a surface. The surface modifications of silicon wafer in various steps led to significant increase in the thickness and the results presented in the Table 1 with standard deviation obtained after measuring at three different locations of the sample. Cauchy model was considered to fit the ellipsometric plot obtained after modification of Si/SiO₂.

The thickness of the hydroxylated Si surface modified with APTES (step 1) yielded silicon wafer with amino groups showing 7.80 Å which is nearly equal to length of the single molecule. Consequent reaction of Si amine surface with DBCO (step 2) resulted in a thickness of 6.50 Å, confirming the amide bonding of DBCO to silicon wafer amino groups. The theoretical length of OT was found to be 29.50 Å and a similar value was found after the attachment of OT to DBCO attached to the silicon wafer (step 3) to yield OT-wafer. This value (33.40 (±0.55) confirmed the bonding of OT to DBCO attached to the silicon wafer via click chemistry.

TABLE 1
Ellipsometric thickness of the various
layer assembly steps of OT-wafer.

| Layer (step #)^{a} | Thickness (Å)^{b} | |
|---|---|---|
| Wafer/SiO2 | 25.05 | (±0.83) |
| Wafer-NH2 (step i) | 7.80 | (±0.34) |
| Wafer-DBCO (step ii) | 6.50 | (±0.62) |
| OT-Wafer (step iii) | 33.40 | (±0.55) |

^{a}The step # are the same steps shown in FIG. 1 applied to silicon wafer.
^{b}The values in the parentheses indicate the RSD values based on three replicate measurements.

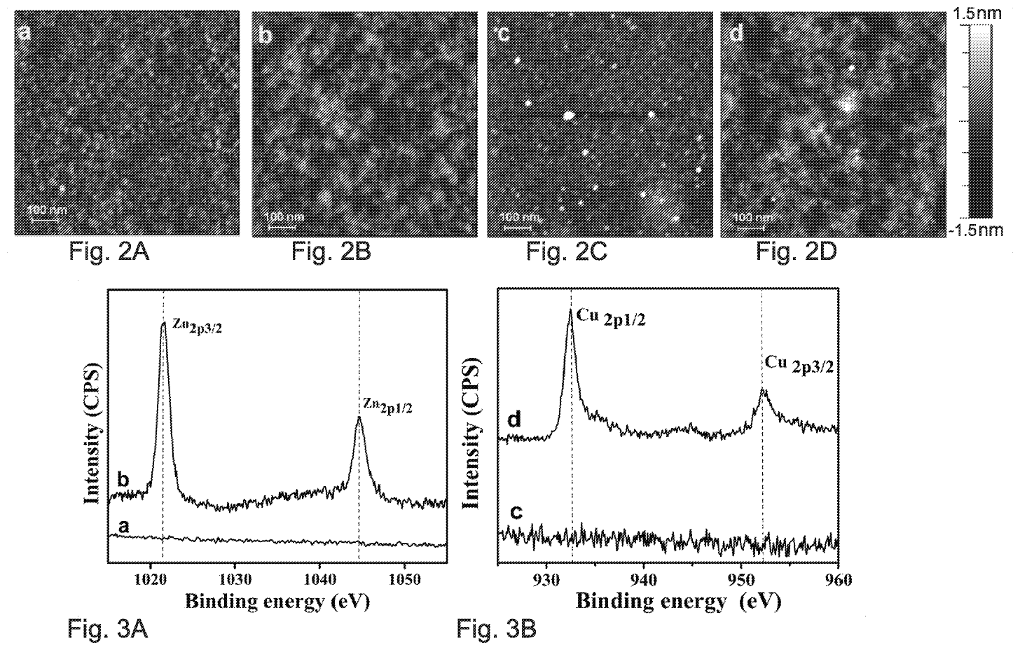

1.2.2 Atomic force microscopy (AFM) of OT-Wafer The variation in mean roughness of the silicon wafer surfaces on each modification step was monitored using atomic force microscopy over surface area 1 μm×1 μm and the obtained topographic images are shown in FIG. 2. Averaged value of root mean square (RMS) of roughness (ρ) was considered to eliminate local effects. Si substrate with hydroxyl functional groups after cleaning using the RCA method showed surface roughness of 2.03 Å, a value that confirms the effectiveness of cleaning protocol.

After modification with 2% APTES, the Si substrate showed a homogeneous surface with a roughness of ˜2.29 Å nm due to aminopropyl functionality containing siloxane coupling unit. After functionalization with DBCO and OT, the surface roughness increased to 2.56 Å and 2.96 Å respectively. Hence, the increase in surface roughness on each layer was clearly correlated to layer-by-layer functionalization of Si substrate. However, the roughness of the surfaces (ρ<5 Å) indicated homogeneous and continuous monolayers' surfaces in all stages of modification. It is worth mentioning that the roughness of the OT-Sensor was increased to 4.8 Å after incubation of the electrode in 1 nM Zn²⁺ solution (image is not shown) is due to coordination of the metal ion to OT.

1.2.3 X-ray photoelectron spectroscopy (XPS) In order to investigate the chelation of the metal ions to OT, the silicon substrates modified with OT-Wafer before and after incubation with Zn²⁺ and Cu²⁺ were characterized using XPS. As can be understood from FIGS. 3A and 3B, the OT-wafer did not show any peak corresponding to Zn²⁺ and Cu²⁺. However, after incubation with Zn²⁺ ions, the spectrum (FIG. 3A) indicated a peak at 1018.7 eV corresponding to Zn_{(2p3/2)} in 2⁺ oxidation state. This value was lower than the binding energy of fully oxidized zinc due to chelation by OT. The OT-Wafer incubated with Cu²⁺ solution, showed two peaks at 932.6 and 952.1 eV attributed to Cu_{(2p3/2)} and Cu_{(2p1/2)} respectively.

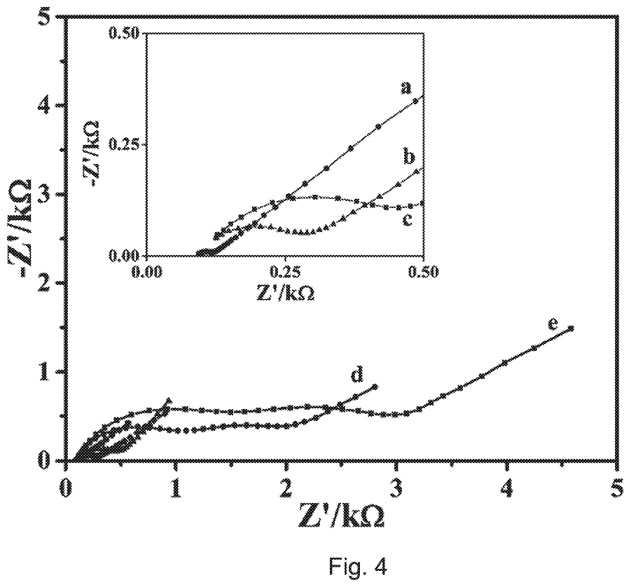

1.2.4 Electrochemical impedance spectroscopy (EIS) of the OT-Sensor EIS is mainly characterized by studying the variation in charge transfer resistance (RCT) at the electrode-electrolyte interface. Generally, EIS spectra of self-assembled monolayers (SAM) formed electrodes are analyzed by fitting the plots with Randles equivalent circuit in which capacitance (C_{dl}) is replaced by constant phase element (CPE). The circuit consists of four elements: (i) the Ohmic resistance of the electrolyte solution (Rₛ) (ii) the interfacial double layer capacitance (C_{dl}) between electrode-electrolyte interface (iii) the electron transfer resistance (R_{CT}) and (iv) the Warburg impedance (Z_{w}) that results from the diffusion of ions from bulk electrolyte to electrode interface. For each measurement, it is important to maintain the same distance between reference electrode and the modified electrode for all the experiments. All measurements have been carried out with 5 mM [Fe(CN)₆]⁴⁻ and [Fe(CN)₆]³⁻ in 0.1 M phosphate buffer solution (PBS) at pH 7.0. Nyquist plots (real Z′ vs. imaginary Z′) obtained for the GCE after each modification step is presented in FIG. 4. The impedance spectra were studied with suitable equivalent circuit to obtain specific elements of resistive and capacitive components and the fitting results are listed in Table 2. The polished bare GCE shows a very low charge transfer resistance of 22.4Ω (±1.4). After the surface was grafted with APTES, the R_{CT} value is increased to 260.1Ω (±3.1) due to hindrance of electron transfer kinetics from the non-conductive layer. Following the condensation of DBCO to the alkylamine functionalized wafer, the R_{CT} value increased to 438.7Ω (±12.7) due to the addition of the aromatic hydrophobic group. Subsequent to the click addition of OT-N₃ we observed an increase in the R_{CT} value to 803.6Ω (±2.6) and an additional semicircle at 1281Ω (±2.8) that appears in higher Z′ range in the Nyquist plot. The increase in charge transfer resistance is due to the increase in the insulating layer thickness that results from the addition of OT to the surface. The molecular explanation for the lower frequency semicircle resistance and capacitance will be discussed later.

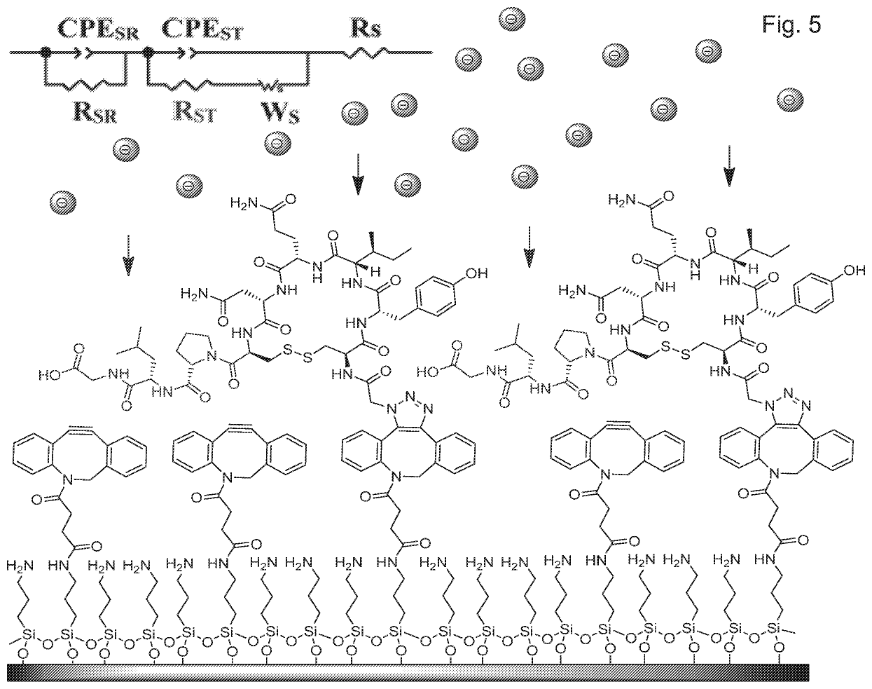

As is seen in the FIG. 5, the Nyquist plot of OT-Sensor is a combination of two interfaces (semicircles). Following models of electrode/electrolyte interfaces has been used to describe the physical origin of the Nyquist plots. The equivalent circuit for as-immobilized OT on GCE is constructed from the following elements: the Ohmic resistance of the electrolyte solution Rₛ, Warburg impedance, R_{w} (contributed to diffusion of ions bulk electrolyte to electrode interface), two capacitive layers; one is due to the OT-Ring/electrolyte interface (C_{RS}) and another, that is due the OT-Tail/electrolyte interface (C_{TS}) with corresponding two electron transfer resistances R_{RS} and RTS respectively (FIG. 5B).

The equivalent circuit in the insert of FIG. 5 represents the circuit that best fits to the impedance data for the OT-Sensor. The anchoring of OT molecule onto GCE-DBCO provided two capacitive elements and consequently the electrode/electrolyte consisted of two interfaces, RS and TS in series. It is assumed that it results from the two domains in the monolayer one is ring dominated domain and the other tail dominated domain.

Each assembly step results with an increase in the monolayer's capacitance. Exposing the OT-Sensor to Zn²⁺ resulted in a significant increase of the impedance.

TABLE 2
Equivalent circuit elements fitted values for the OT-Sensor of FIG. 4

| Step | Rₛ (Ω · cm²) | C (μF cm⁻²) | | R¹_{ct} (Ω · cm²) | | R²_{ct} (Ω · cm²) | CPE (μF cm²) | R_{w} (Ω · cm²) | | χ² |
|---|---|---|---|---|---|---|---|---|---|---|
| Bare GCE | 94.4 (1.3) | 0.91 | (0.52) | 22.3 | (1.3) | — | — | 353.7 | (0.1) | 0.013 |
| GCE-NH2 | 95.8 (1.4) | 29.78 | (2.54) | 260.1 | (3.0) | — | — | 689.5 | (1.5) | 0.039 |
| GCE-DBCO | 96.5 (1.2) | 33.67 | (1.97) | 438.6 | (12.7) | — | — | 442.8 | (1.6) | 0.018 |
| OT-Sensor | 95.9 (1.9) | 45.31 | (1.26) | 659.2 | (20.4) | 1430 (21) | 10.2 (2.2) | 462.7 | (16.4) | 0.011 |
| 1 nM Zn2+ | 96.4 (1.5) | 46.25 | (1.18) | 812.6 | (14.7) | 2157 (32) | 49.5 (1.6) | 542.4 | (10.6) | 0.015 |

1.3 Impedimetric Detection of Zn²⁺/Cu²⁺ Ions by the OT-Sensor

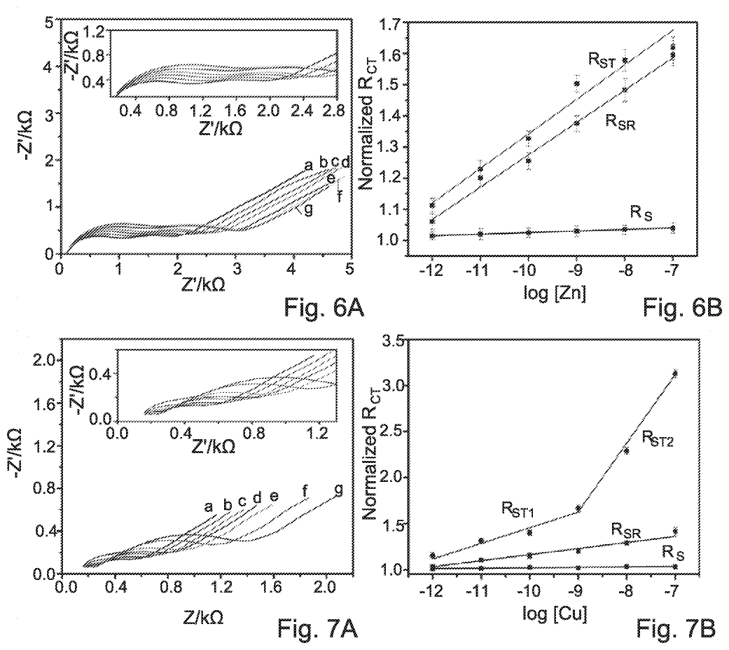

Preliminary studies confirm that the presence of metal ions (Zn²⁺) result in an increase of the impedimetric signal. To evaluate the correlation between metal ions concentration and the impedimetric signal, a series of experiments were performed in which the OT-sensor was exposed to increasing concentrations of either Zn²⁺ or Cu²⁺ before the impedance was recorded. OT-Sensor was exposed to Zn²⁺ concentrations in a range of 1 μM to 100 mM and the impedance was measured and modeled. The analysis showed a gradual increase in impedimetric signal in response to the increase in Zn²⁺ concentration (FIG. 6A). The two semicircles grows in diameter monotonically with the increase in concentration while the slop of the linear part remains constant. It is assumed that the increase in the charge transfer resistance is related to the increase in OT-Zn²⁺ chelation that results from exposure to higher concentration of metal ions. The diffusion constant of the redox active species does not change with the increase in the analyte concentration (see FIG. 6B, Rₛ). The two OT monolayers' resistance components responds in a similar way to Zn²⁺ concentration, R_{ST}=0.11 dec⁻¹ and R_{SR}=0.10 dec⁻¹.

Normalized R_{CT} is defined as the ratio of charge transfer resistance for the concentration of M²⁺ (R_{CT}(Cᵢ)) and charge transfer resistance of blank solution (R_{CT}(Cₒ)) of the OT-sensor. Normalized R_{CT} is plotted against Zn²⁺ concentration (FIG. 6B) and shows good linear correlation of (R_{CT}(Cᵢ)/R_{CT}(Cₒ)=0.104 log [Zn²⁺/M]+2.314) over a range of Zn²⁺ concentration from 1 μM to 100 mM with a regression coefficient of 0.989. The slope of the fitted curve refers to the sensitivity of the sensor and found to be for R_{ST}˜0.10 M⁻¹. Full analysis of the other two resistors in the equivalent circuits shows that R_{SR} has similar sensitivity to R_{ST} R_{SR}˜0.11 M⁻¹ and the change in ion concentrations has negligible effect on the solution's resistance, R_{S}˜0.005 M⁻¹.

OT-sensor was exposed to Cu²⁺ concentrations in a range of 1 μM to 100 mM and the impedance was measured and modeled. The analysis showed a gradual increase in the charge transfer resistance in response to the increase in Cu²⁺ concentration (FIG. 7B). The plot of normalized charge transfer resistance against the logarithm of Cu²⁺ represents a linear equation; R_{CT}(Cᵢ)/R_{ct}(Cₒ)=1.82+0.065 log [Cu²⁺]. This indicates that there is a linear correlation between Cu²⁺ concentration and R_{SR} with a slope of 0.065 M⁻¹ similar to the R_{SR} for Zinc ions. Rs for the two ions are also similar. Contrary to the linear correlation observed for the R_{ST} resistance component in response to the increase of Zn²⁺ concentration, here we observed two linear regimes for R_{ST}: R_{ST1} for the pM-nM and R_{ST2} for the nM-mM concentrations range. The slope of the fitted curve for the low concentration regime was found to be R_{ST1}˜0.16 M⁻¹, similar to the response for zinc ions. The high concentration regime shows a much striper slope R_{ST2}˜0.72 M⁻¹.

The slope of the R_{SR} in response to Zn²⁺ concentration is stiper than the R_{SR} slope recorder for the response to Cu²⁺. This indicate a slightly better sensitivity of the OT-Sensor toward Zn²⁺ compared to Cu²⁺ in the lower concentration range (pM-nM). In this range R_{ST1} for Cu²⁺ and R_{ST} for Zn²⁺ are similar. Interestingly the R_{ST2} slope for Cu²⁺ is significantly stiper than that of the corresponding R_{ST} recorded in response to Zn²⁺ concentration in the nM-mM concentrations range. This results in a better signal strength for the high concentration range from 66-213% vs. 50-61% for copper ions versus zinc ions.

It is suggested that the two hemicircles corresponds to two different domains in the OT monolayer—the first domain is rich with the ring motif (see FIG. 5) and the major component of the second domain is the OT tail (see FIG. 5). Each domain has a different affinity towards metal ions. In both dose response experiments we observed a different behaviour in the plot. While in response to Zn²⁺ the increase of the first hemicircle is more profound than that of the second hemicircle, in response to Cu²⁺ concentration the increase in the second hemicircle is more dominant. Many previous reports describe oxytocin as having two metal binding regions, the first being the ring itself and the second being the tail. These reports claim that while Cu²⁺ complex OT in an tetrahedral conformation mostly through the amides of the tail, Zn²⁺ forms octahedral complex with OT through the carbonyls of the ring. It is assumed that the different behaviour of OT-Sensor toward Zn²⁺ and Cu²⁺ is related to the nature of the binding of free OT to these metals as reported previously.

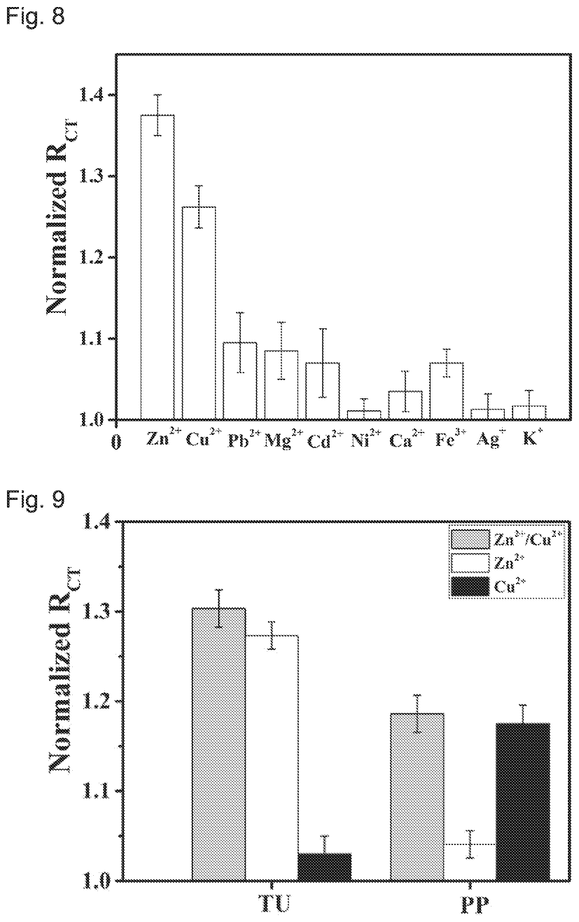

Selectivity studies The selectivity of the OT-Sensor towards Zn²⁺ and Cu²⁺ was investigated from the response of the sensor to various additional metal ions including Pb²⁺, Mg²⁺, Pb²⁺, Cd²⁺, Ni²⁺, Ca²⁺, Fe³⁺, Ag⁺ and K⁺. These ions are known to frequently co-exists with Zn²⁺ and Cu²⁺ in biological and environmental systems. The histogram of normalized charge transfer resistance of each metal ion is depicted in FIG. 8 and in the S The sensor shows higher response to Zn²⁺ followed by Cu²⁺ in comparison to other metal ions. It may be assumed that the selectivity of the sensor towards Zn²⁺ and Cu²⁺ is due to ionic size, charge and chelating properties of OT.

Selective determination of Zn²⁺ and Cu²⁺ The OT-Sensor showed superior detection of Cu²⁺ and Zn²⁺ compared to other metals. However, it was crucial to determine if the OT-Sensor is capable of detecting Cu²⁺ in the presence of Zn²⁺ and vice versa. The parallel detection of Zn²⁺ and Cu²⁺ was achieved using selective masking strategy. Thiourea (TU) was used to mask Cu²⁺ to enable selective Zn²⁺ detection. Pyrophosphate (PP) was used for masking Zn²⁺ to enable selective Cu²⁺ detection. In order to determine the efficiency of the masking agents on the OT-sensor response, each masking agent was added to the OT-Sensor containing either only Zn²⁺ or Cu²⁺. The results showed that negligible response for Cu²⁺ in the presence of TU in contrast to Zn²⁺ that showed full response. Similarly, when the sensor response was recorded for the mixture and individual ions in presence and absence of PP, the results showed preferential masking of Zn²⁺ by PP. These studies indicated that preferential masking of Cu²⁺ and Zn²⁺ within a mixture can be attempted. Studies using a 1:1 mixture of Cu²⁺ and Zn²⁺ showed that charge transfer decrease in the presence TU compared to the mixture without TU and reached similar level of response observed when only Zn²⁺ was used (FIG. 9). When PP was added to the 1:1 mixture of Cu²⁺ and Zn²⁺, a decrease in charge transfer was observed and reached the same level of response as was recorded for the solution containing Cu²⁺. These results showed that the OT-Sensor can be used for the selective detection of Zn²⁺ and Cu²⁺ even when both ions are present in the mixture simple by masking one of them selectively.

Discrimination of Zn to Cu Ratio in Healthy and MS (Multiple Sclerosis) Sera Samples

The Zn²⁺ to Cu²⁺ ratio (ZCR) in MS patients is lower than for healthy subjects hence, can be used as a biomarker to detect MS. It is of high relevance to prepare a sensitive and selective electrochemical sensor to enable a fast determination of ZCR in biofluids. In order to evaluate the potential applicability and analytical reliability of the OT-sensor in biofluids, the sensor was used to determine the ZCR in healthy and MS sera samples. For the simultaneous detection of Zn²⁺ and Cu²⁺ in the same sera samples, TU and PP were used to mask one of the metal-ion in the presence of the other.

To determine the ZCR using our OT-sensor, the impedimetric signal was measured of both healthy and MS patients with either TU or PP. The obtained impedimetric signal was normalized and fitted to the calibration curve to determine the concentration of each ion. In case of Cu²⁺ determination, the curve corresponding to R_{ST} or R_{SR} was used as very less difference in results are obtained. Our study indicated that there was a significant reduction of the ZCR value between healthy and MS patients. While the ZCR of healthy patients sera was 20.41, the ZCR value of MS patients sera was 7.46.

The quantification of the metal-ions concentration in the same sera samples was validated using inductively coupled plasmon-mass spectroscopy (ICP-MS). Similar concentrations of both ions were obtained by EIS and ICP-MS validating the method (Table 3). The ZCR calculated from ICP-MS was 26.5 and 6.10 for healthy and MS patients, respectively. These values are in par with the values obtained the OT-Sensor measurements (20.41 and 7.46, respectively). This validates the accuracy of the OT-Sensor in the detection of Zn²⁺ and Cu²⁺ ions in real samples. This proves that the OT-Sensor enable to determine the ZCR in serum in short time and high accuracy.

TABLE 3
Analysis of metal-ions concentration in healthy and multiple sclerosis
(MS) sera samples (These values are expressed as mean values
and the ± RSD values are based on three measurements).

| Device | | | | |
|---|---|---|---|---|
| Sera | EIS of OT-Sensor^{b} | | ICP-MS | |
| sample | Zn²⁺ [M] | Cu²⁺ [M] | Zn²⁺ [M] | Cu²⁺ [M] |
| Healthy | 9.33 × 10⁻⁹ | 4.57 × 10⁻¹⁰ | 2.65 × 10⁻⁹ | 1.00 × 10⁻¹⁰ |
| | (±2.76) | (±3.58) | | |
| MS | 3.88 × 10⁻¹⁰ | 5.20 × 10⁻¹¹ | 5.46 × 10⁻¹⁰ | 8.95 × 10⁻¹¹ |
| | (±4.26) | (±3.65) | | |

^{b}In EIS experiments, Zn²⁺ values were measured in the presence of TU and Cu²⁺ values were measured in the presence of PP.

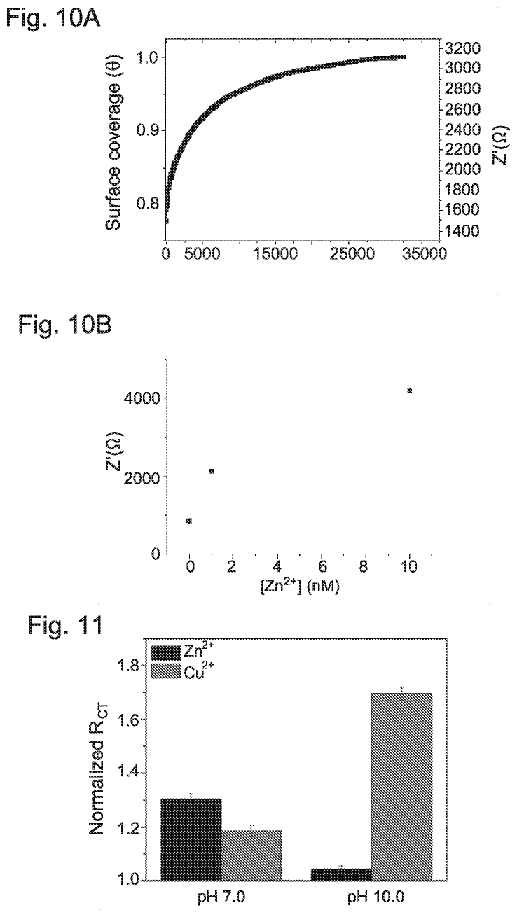

Somatostatin was also tested. The native disulfide bond of the cyclic peptide was utilized for self-assembly on gold electrode. The adsorption kinetics was monitored by AC impedance measurements (FIG. 10A). In addition, the impedometric response to Zn⁺² recognition event at 1.0 nM concentration was evident (FIG. 10B). These preliminary results indicates that the peptide neurotransmitter is an efficient ionic-receptor on gold electrode. The electrochemical system is capable in measuring metal ion binding to surface anchored biopolymers.

N-Methylated Peptides

N-methylated and other N-alkylated peptides utilized in accordance with the invention may be prepared according to available procedures.

N-methylated oxytocin analogues include:

Azidoacetic-[MeGly⁹]OT, Az-9-NMe-OT (structure A below);

Azidoacetic-[MeLeu⁸]OT, Az-8-NMe-OT (structure B below);

Azidoacetic-[MeLeu⁸,MeGly⁹]OT, Az-8,9-diNMe-OT (structure C below).

Additional alkylations as well as different sites of alkylation, of various other peptides have been contemplated and formed.

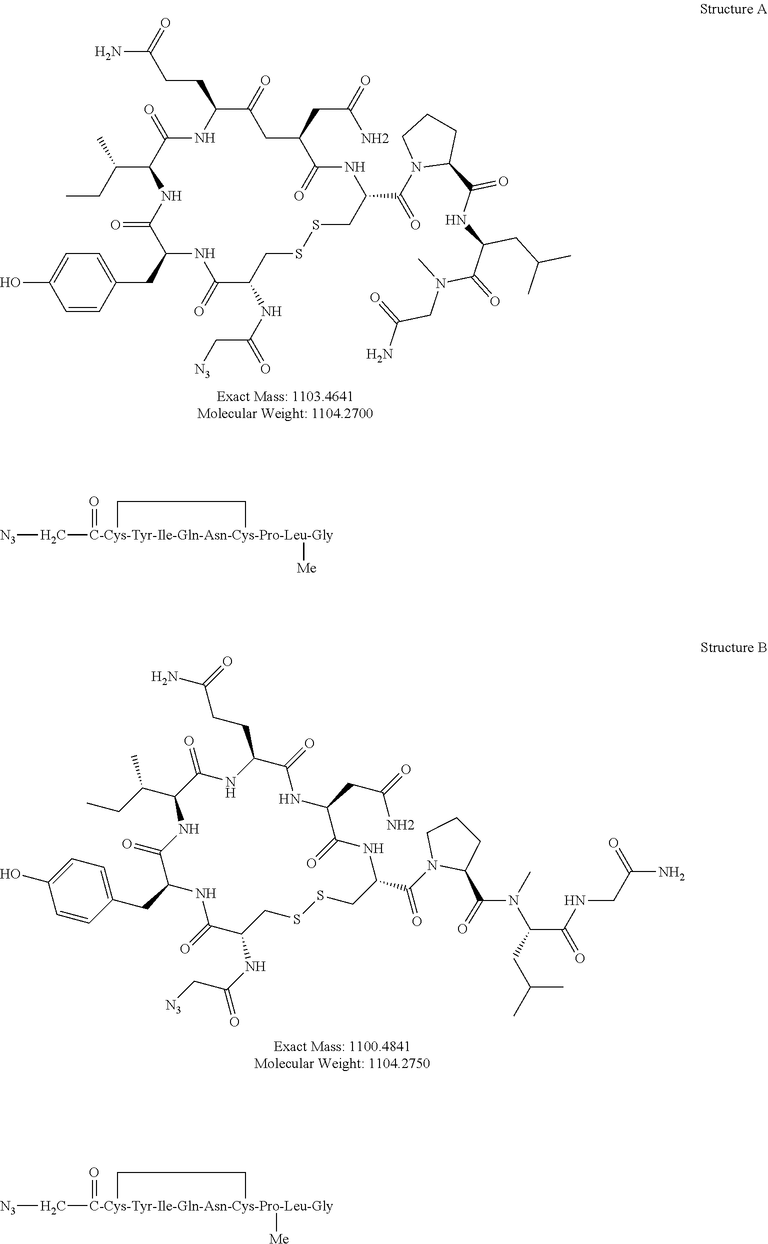

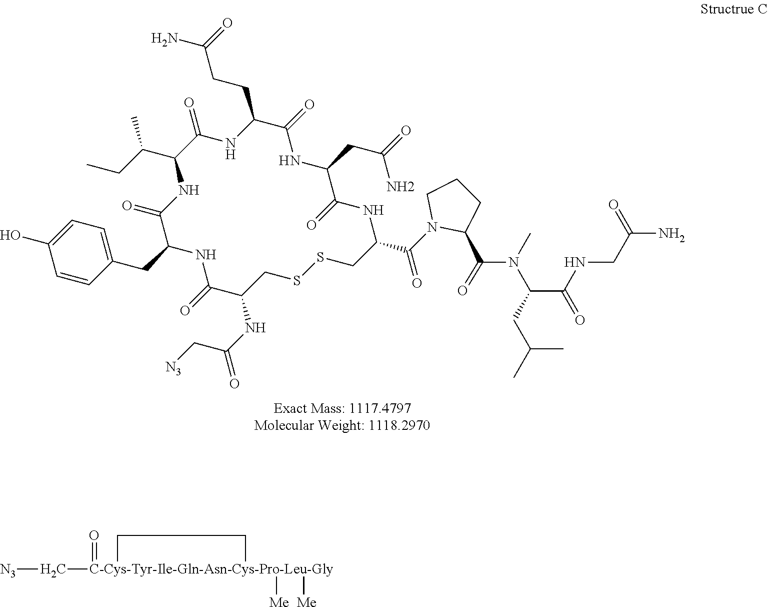

The N-alkylated derivatives provide the opportunity to tailor selectivity and sensitivity to metal chelation by blocking positions involved in binding metal ions, e.g., Cu²⁺ ions in tail part of oxytocin, so that Zn²⁺ ion affinity is not affected.

Peptides were synthesized using standard protocols as described for cholesteryl peptides. The additional procedure was applied in the N-methylated positions as described below.

N-Methylation:

Peptides were N-methylated according to the procedure described in J. N. Naoum et al. Beilstein J. Org. Chem. 2017, 13, 806-816, as shown in the Scheme below:

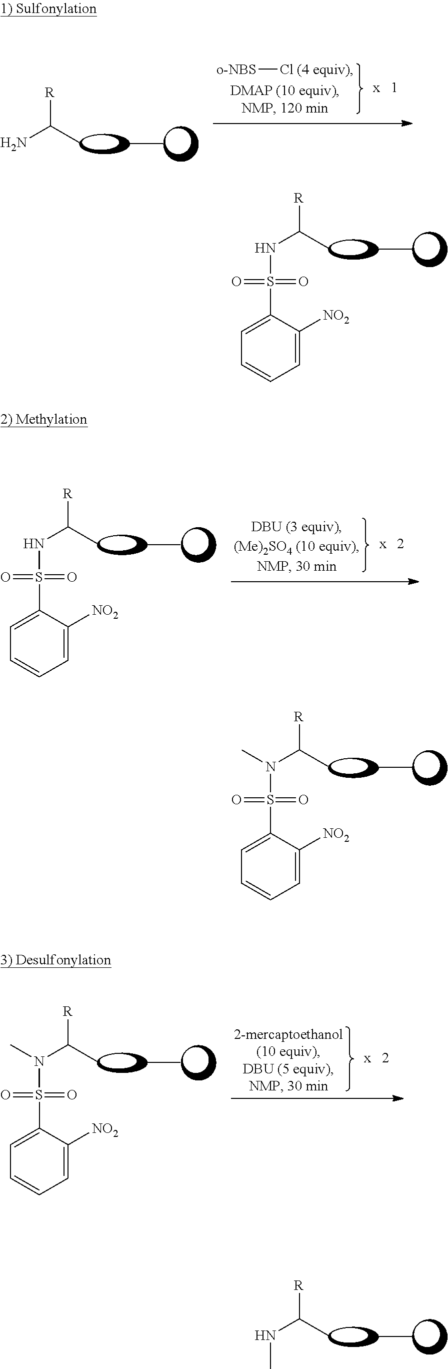

After coupling of the amino acid residue to be N-methylated and Fmoc group removal, the first step was sulfonylation by introduction of the o-nitrobenzenesulfonyl (o-NBS-Cl) to primary amine in the presence of amine, here 4-dimethylaminopyridine (DMAP), so the semi-protected sulfonamide can undergo a selective mono-methylation. The next step was methylation performed by incubation of sulfonylamide with (Me)₂SO₄ in the presence of 1,8-diazabicyclo[5,4,0]undec-7-ene (DBU). Finally the o-NBS was removed using a combination of 2-mercaptoethanol and DBU. Methylation and desulfonylation steps were repeated twice.

After completion of N-methylation procedure the peptide chains were elongated and followed the procedure and analyses as described for cholesteryl peptides.

Variation in Metal Binding Sensitivity Before and after N-Methylation of a Peptide Used in Accordance with the Invention—the Oxytocin Example

It was observed that oxytocin (OT) based biosensor is highly sensitive to the Zinc(II) and Copper(II) ions with a difference in binding motifs of OT. In case of Zn²⁺ ion, the ion binds to ring and tail parts of OT equally where as in the case of Cu²⁺ ion the tail part is highly sensitive in comparison to the ring part. In order to achieve the selective Zn²⁺ ion sensor, N-methylation was used on the glycine in the tail part of OT. The N-methylation helps in preventing chelation of Cu²⁺ as the compound could not form anionic nitrogen.

The effect of pH on the selectivity was further evaluated. Impedimetric studies of OT-GCE after incubating in 1 nM Zn²⁺ or Cu²⁺ both at pH 7.0 and at pH 10.0 have been carried out and are summarized in (FIG. 11). The sensor signal is presented as the normalized charge transfer resistance (R_{CT}) which is calculated as ratio between R_{CT} for the concentration of M²⁺ (R_{CT}(Cᵢ) and blank solution (R_{CT}(Cₒ)) of the sensor. The study shows that at pH 7.0 there was a significant response of the OT-sensor both to 1 nM Zn²⁺ as well as to 1 nM Cu²⁺. However, the response of OT-sensor towards Cu²⁺ and Zn²⁺ at pH 10.0 was very different. At pH 10.0, no significant signal was observed in the presence of 1 nM Zn²⁺. In contrast, the response for 1 nM Cu²⁺ was enhanced in comparison to pH 7.0.

The results provide strong evidence to the hypothesis that the metal detection may also be governed by the binding mechanism. Since the ligation of Zn²⁺ to OT takes places through carbonyl oxygens while Cu²⁺ chelation of OT takes place through deprotonated nitrogens of amide the pH plays a crucial role in the binding. At pH 10.0, the deprotonation of amides is more favorable, hence, increases the affinity towards Cu²⁺. It may be assumed that the lack of affinity towards Zn²⁺ at pH 10 results from the preferred formation of zinc hydroxide and the conformational changes associated with the electrostatic repulsions of the deprotonated amides.

After the confirmation of selective detection of Cu²⁺ at pH 10.0 using OT sensor, the sensor response towards various Cu²⁺ concentrations has been studied. The OT sensor was exposed to increasing concentrations of Cu²⁺ ranging from 1 μM to 0.1 μM and the corresponding impedance spectra were recorded.

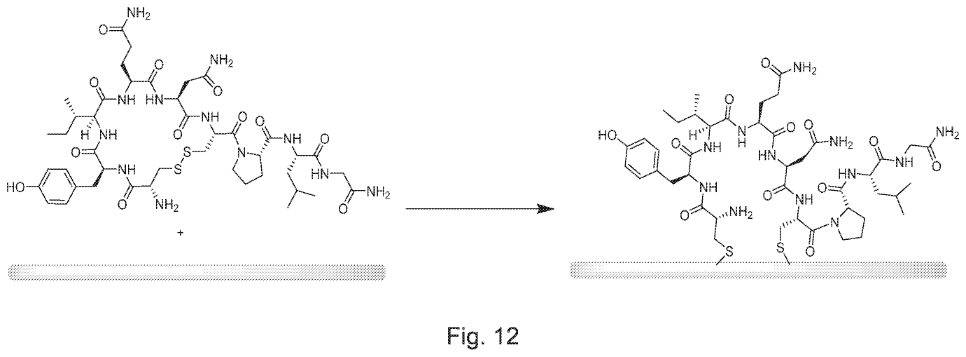

Alternatively to the multistep assembly demonstrated in FIG. 1, oxytocin based metal sensing on gold surface was also achieved by direct assembly of the peptide on the surface (Au-OT sensor), as shown in FIG. 12.

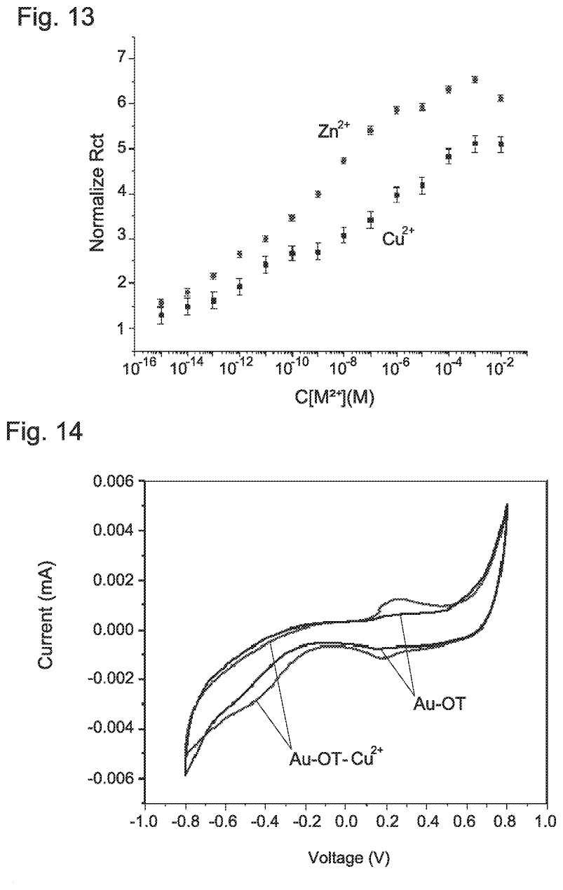

Copper and Zinc ions dose response using EIS: The dose response represented in FIG. 13 as normalized R_{CT} vs exposure to metal ion solution. Normalized R_{CT} presents calculation the ratio of R_{CT} for the OT sensor exposure to concentration of M²⁺ (R_{CT}(C)) and Rct value of Au-OT sensor before exposure to metal ions.

Cyclic Voltammetry analysis of OT layer with Cu²⁺ presence: By the Cyclic Voltammetry analysis one can recognize Cu²⁺ oxidation peak at 0.26 V (FIG. 14), which is significantly shifted from oxidation peak of free Cu²⁺ ion (0.12 V). This may be attributed to OT-Cu complexation. By mathematic calculations, the density of the ions in the layer (0.32 ions/nm2). In addition, the calculated ratio of OT: Cu²⁺ on the surface is 1:1.5.

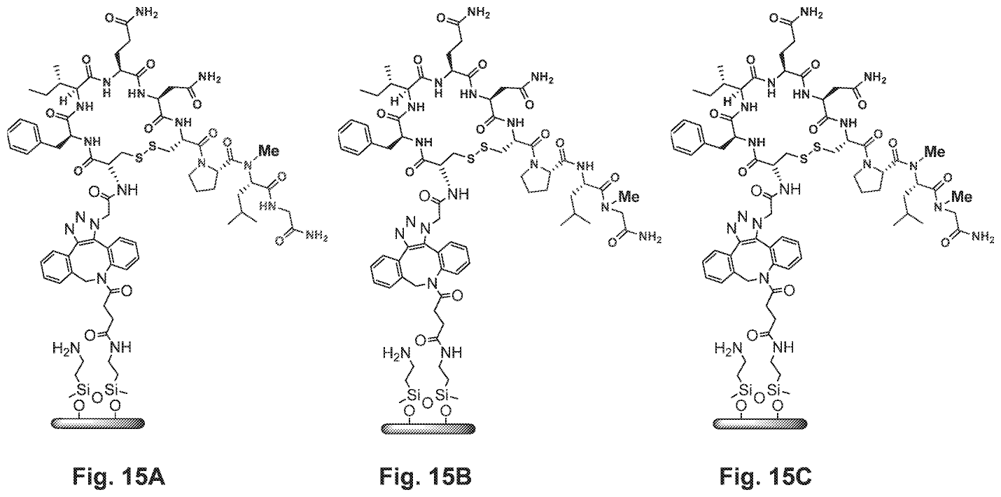

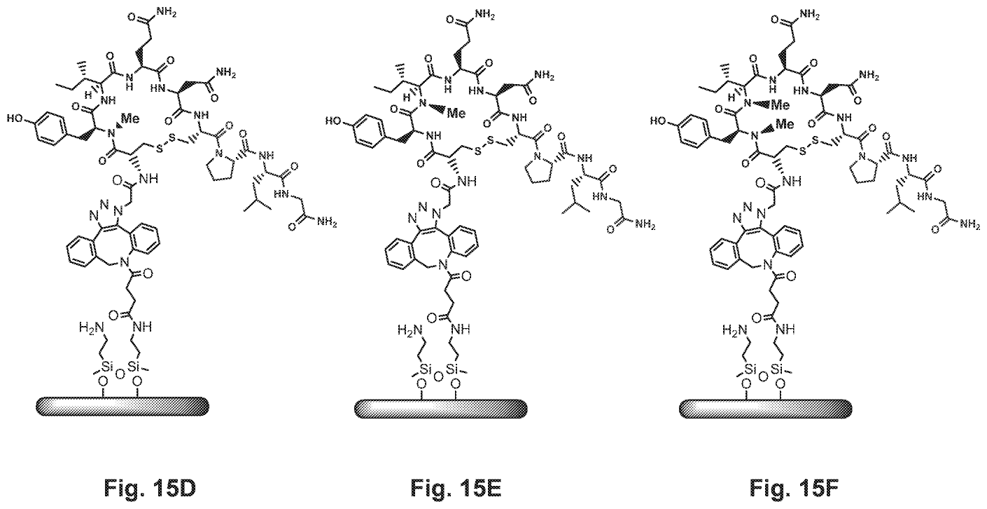

As demonstrated herein, the OT based biosensor is highly sensitive toward both Zinc(II) and Copper(II) ions. In case of Zn²⁺ ion, the ion binds to ring and tail parts of OT equally whereas in the case of Cu²⁺ ion the tail part is highly sensitive in comparison to the ring part. In order to achieve the selective Zn²⁺ ion sensor, N-methylation on the tail part of OT (N-methyl Gly(8) OT, N-methyl Leu (9) OT and di-N-methyl Gly-9,Leu-8-OT) was tested. The N-methylation of OT aimed for preventing chelation of Cu²⁺ as the compound could not form anionic nitrogen and allow for specific zinc sensing. FIGS. 15A-F show structures of N-methyl (8) OT, N-methyl (9) OT and N-methyl (8,9) OT.

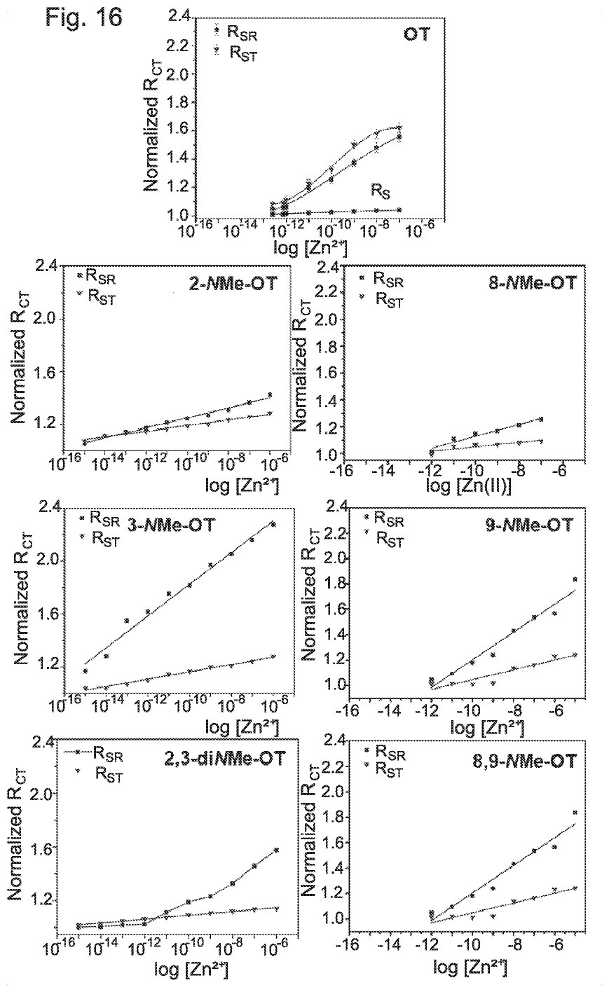

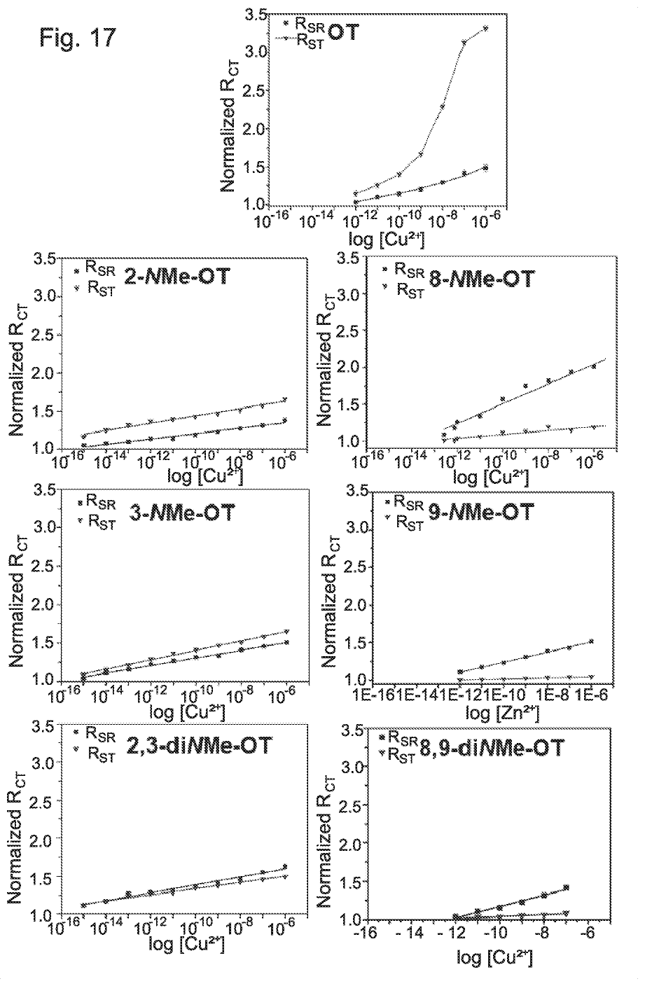

N-methylated OTs has been immobilized on the glassy carbon electrode using click chemistry and tested for the faradaic impedimetric detection of Zn²⁺/Cu²⁺. Electrochemical impedance studies were performed for the detection of Zn²⁺ in different concentrations. The normalized R_{CT} value was considered as the ratio of R_{CT} in the absence of metal ion to R_{CT} in the presence of metal ion (FIGS. 16A-D, FIGS. 17A-D).

8-N-Me-OT (FIGS. 16A-D) did not provide selectivity between the two ions and the sensitivity to both ions was low. 9-NMe-OT (FIGS. 17A-D) showed that the tail part of OT was completely irresponsive to the presence of Copper while its zinc binding was maintained. 8,9-diN-Me-OT showed that the dimethylated OT response to copper was almost completely gone both for the tail and the ring.

As is observed from the calibration curve of 8-N-Me-OT for Zn²⁺, the sensor response starts from 1 nM Zn²⁺. However, there is a preferred response to Cu²⁺ from in the very low concentrations. It is interesting to observe the very high response to Zn²⁺ from the 9-NMe-OT. The normalized R_{CT} is almost similar from the both ring and tail parts. Even though there is significant response from R_{SR} to Cu²⁺, but no or negligible response was observed from the R_{ST}. From the 8,9-diNMe-OT OT modified GCE shows significant response to Zn²⁺ from the tail in comparison to ring domain. It is obvious that there is no response to Cu²⁺ as the tail part of OT is methylated on two sites.

In order to easily distinguish the selective sensing of Zn²⁺, a histogram of the different methylated OT sensors response to 0.1 μM Zn²⁺/Cu²⁺ has been provided. N-methylated (9) OT with glycine showed selectivity towards Zn²⁺ with suppressed Cu²⁺ response. In addition, the response from the ring and tail domains toward zinc has similar sensitivity over the range of 10⁻¹⁵ M to 10⁻⁶ M. The selectivity of 9-NMe-OT to Zn²⁺ as compared to Cu²⁺ imply that the methylation of the glycine prevented the deprotonation required for copper complexation. 8-NMe-OT showed low response to Zn²⁺ and a slightly better response to Cu²⁺. The 8,9-diNMe-OT response of the tail and the ring part was metal ion dependent.

OT was further anchored to a gold surface by linking it to mercaptooctanoic acid (MOA) that is self-assembled on gold surface. In this case, there was no possibility to bind Cu²⁺ as it has no primary amine which can act as the primary chelation ligand prior to the cascading of deprotonation. The Au-MOA-OT sensor showed linear increase in charge transfer resistance (R_{CT}) with increase in concentration of Zn²⁺ over a range from 10⁻¹⁴ M to 10⁻⁸ M with a detection limit of 3.2×10⁻¹⁴ M. Further, non-faradaic impedimetric studies were conducted at single frequency and confirm the signal arising from the Zn²⁺ addition.

FIG. 18 presented a shematic representation of step-wise anchoring of OT on Au surface: (A) self-assembling of MOA (B) coupling of OT to MOA through amide bond and (C) passivation with 6-mercaptohexanol.

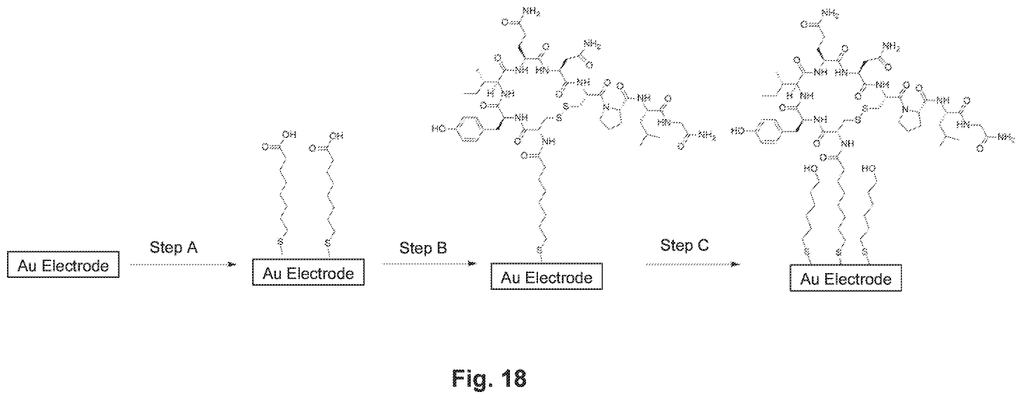

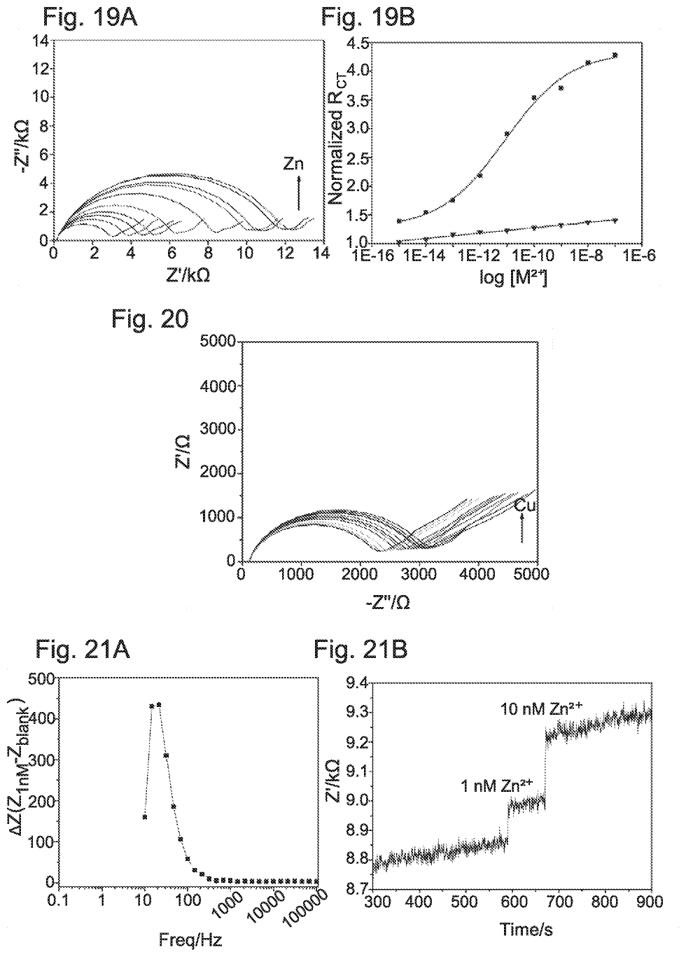

The electrochemical impedance detection of Zn²⁺ and Cu²⁺ has been carried out by immersing into the respective concentration for five minutes and EIS has been performed to obtain Nyquist plots. The normalized R_{CT} was determined from the ratio of R_{CT} of the sensor exposed to M²⁺ concentration to R_{CT} of the sensor in blank solution (FIG. 19A).

FIG. 19B illustrates the sigmoidal relationship between the normalized R_{CT} and logarithmic concentration of Zn²⁺ ion of the Au-MOA-OT sensor. The data were fitted to Hill equation 1, which is a typical binding model of a biosensor response: [1].

$$Normalized {R}_{CT} = {R}_{CT , lim} + \frac{{R}_{CT , 0} - {R}_{CT , lim}}{1 + {( \frac{{C}_{Zn}}{{K}_{D}} )}^{h}}$$

The Au-MOA-OT sensor showed high sensitivity towards Zn²⁺ in comparison to any other metal-ions including Cu²⁺. The proposed high sensitive and fast-responding OT self-assembled sensor for Zn²⁺ here can open new avenues for the development of point-of-care devices and clinical sensors.

The change in impedance spectra with respect to Cu²⁺ was studied at various concentrations of Cu²⁺. The Nyquist plots for Cu²⁺ and the corresponding calibration plots have been presented in FIGS. 20A-B, respectively.

It is clear from the plots, that the Au-MOA-OT sensor has shown a very low sensitivity towards Cu²⁺. The studies reveal that OT has two ligation sites or domains; ring part and tail part. In case of Zn²⁺ binding, OT approaches near octahedral structures by ligation through carbonyl oxygens of both ring and tail parts. In contrast, OT forms square planar complex with Cu²⁺ mostly through the amide nitrogen of the tail domain. In this way, the ring and tail binding sites involved in the detection of Zn²⁺ chelation whereas only the tail binding site involved in Cu²⁺ chelation.

The increase in impedance due to Zn²⁺ binding to OT is further directly proved from the non-faradaic impedance studies conducted in the absence of any redox species. Time-dependent change of real impedance (Z′) with the addition of Zn was studied in the plain ammonium acetate buffer without containing any redox probe. The optimized frequency to be applied in the non-faradaic impedance spectroscopy was determined from the change in real part of Z before and after addition of zinc-ions (FIGS. 21A-B).

As suggested by FIG. 21A, the maximum change in the impedance was observed in the range of 17.5-20 Hz. Real-time measurements of real Z were carried out at 20 Hz frequency. When the sensor is reached equilibrium after 300 seconds, Zn²⁺ concentration has increased by adding suitable aliquots into the cell. It causes a prompt increase in Zᵣₑₐₗ over 5 seconds time scale followed by a very slow increase in Zᵣₑₐₗ.

From these results, it can be emphasized that the Au-MOA-OT sensor is highly sensitive to Zn²⁺ ion and opens an avenue to develop a biosensor for Zn²⁺ detection.

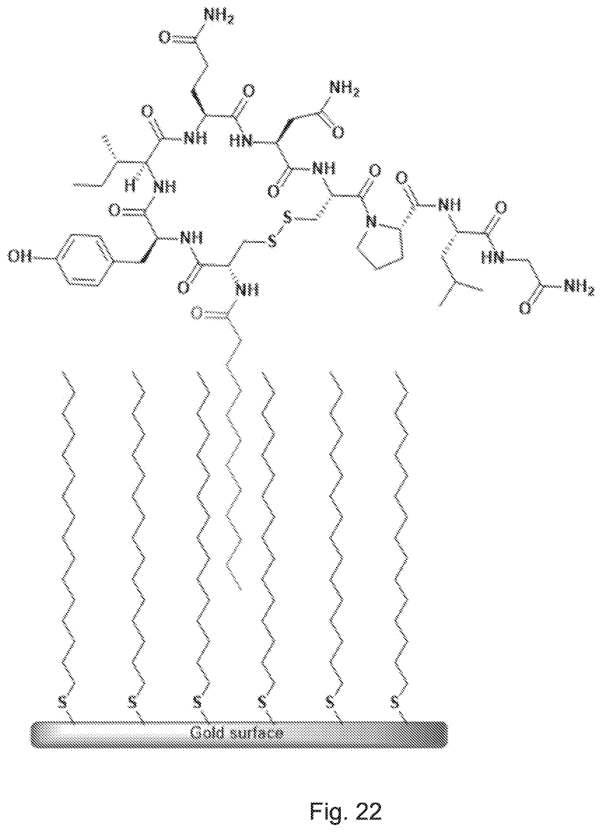

The OT membrane model, Au-lip-OT sensor (FIG. 22), further based on a new concept, in which Zn²⁺ ions is sensed with dodecanoic modified Oxytocin that is not covalently bonded to the surface. After optimization of the self-assembled monolayer of hexadecanethiol, it was concluded that only impedance value higher or equal to 90 kΩ indicates the presence of a dense monolayer. These high values are due to the density of the monolayer, and it is an essential condition to allow the dodecanoic-Oxytocin to integrate into the monolayer.

This alternative method provides highly dense monolayers, high impedance compared to other types of surface modifications, the sensing molecule is not covalently attached to the surface, the sensing molecule keeps the native conformation, this sensing strategy would allow for specific sensing of zinc and not copper, and this method may have further implications for using membrane bound molecules for sensing.

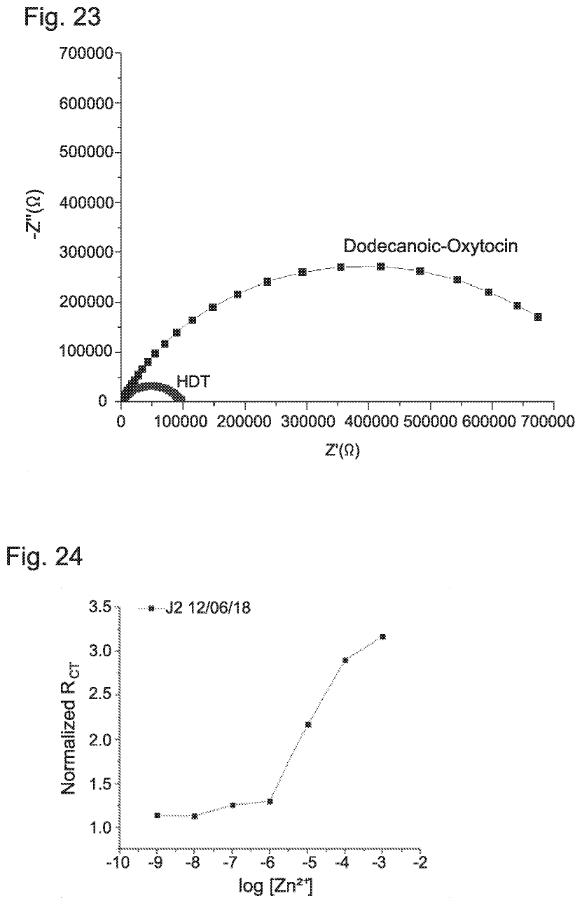

As shown in FIG. 23, the OT-hexadecane thiol was highly dense.

TABLE 4

| J2 Electrode | R_{CT} | |
|---|---|---|
| Bare gold electrode | 119 | Ω |
| Hexadecanethiol modified | 342 | kΩ |
| gold electrode | | |
| Wash with buffer | 563 | kΩ |

FIG. 23 shows an increase in the value of the impedance after incubation of the electrode into Dodecanoic-Oxytocin solution. This result, and the dose response obtained (FIG. 24) proves that Zn²⁺ ions can be sensed with a fluid system, in which the OT is not covalently bonded to the surface.

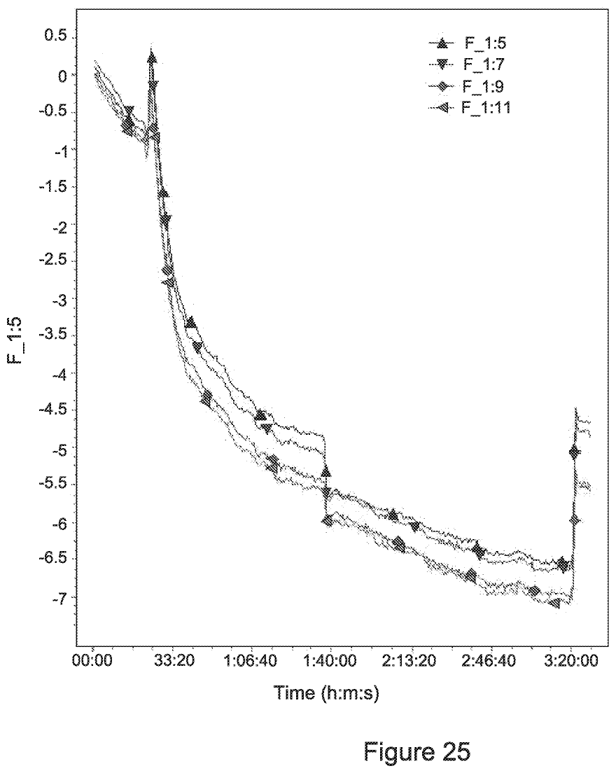

FIG. 25 shows that the frequency decreases with time during about 12 hours. The first hour corresponds to adsorption of hexadecanethiol on the surface, and the next hours correspond to reorganization of hexadecanethiol in self-assembled monolayer on the surface.

Use of Sensors of the Invention in Medicine, Neurodegenerative Disease—Case Study: Zn²⁺ to Cu²⁺ Ratio Determination in Diluted Sera Samples

The Zn²⁺ to Cu²⁺ ratio in Multiple Sclerosis (MS) patients is lower than for healthy subjects and, hence, can be used as a biomarker to detect MS. It is of high relevance to prepare a sensitive and selective electrochemical sensor to enable a fast determination of Zn²⁺ to Cu²⁺ ratio in biofluids. In order to evaluate the potential applicability and analytical reliability of the OT-sensor in biofluids, the sensor was used to determine the Zn²⁺ to Cu²⁺ ratio in healthy and MS diluted sera samples and the results were compared to ICP-MS analysis of the same samples. For the simultaneous detection of Zn²⁺ and Cu²⁺ in the same diluted sera samples, TU and PP were used to mask one of the metal ions in the presence of the other. The study indicated that there was a significant reduction of the Zn²⁺ to Cu²⁺ ratio value between healthy and MS patients. While the Zn²⁺ to Cu²⁺ ratio of healthy patient's sera was 9.11, the Zn²⁺ to Cu²⁺ ratio value of MS patient's sera was around 4-6.

The quantification of the metal-ions concentration in the same sera samples was validated using inductively coupled plasmon-mass spectroscopy (ICP-MS). Slightly higher concentrations of both ions were obtained by EIS due to the other serum components in comparison to ICP-MS (Table 5). The Zn²⁺ to Cu²⁺ ratio in diluted sera samples calculated from ICP-MS for healthy subjects is 5.82±0.05; while this ratio drops to 2.15±0.07 and 2.33±0.01 (with ≤5% RSD) for two different MS patients. By considering the Zn to Cu ratio as an indicator, the values are in par with the values obtained by the OT-Sensor measurements: 9.11±0.08, for the healthy subject and 6.01±0.11 and 4.11±0.07 for the two different MS patients. This proves that the OT-Sensor enable to monitor changes in Zn²⁺ to Cu²⁺ ratio in sera samples as a tool to evaluate patients' health status.

TABLE 5
Analysis of metal-ions concentration in healthy and MS patient's sera samples.

| Sera | EIS of OT-Sensor^{b} | | ICP-MS | | Zn²⁺ to Cu²⁺ ratio | |
|---|---|---|---|---|---|---|
| sample | Zn²⁺ [M] | Cu²⁺ [M] | Zn²⁺ [M] | Cu²⁺ [M] | EIS | ICP-MS |
| Healthy | 7.75 × 10⁻⁸ | 8.50 × 10⁻⁹ | 5.47 × 10⁻⁸ | 9.39 × 10⁻⁹ | 9.11 (±0.08) | 5.82 (±0.05) |
| | (±1.7 × 10⁻⁹) | (±2.6 × 10⁻¹⁰) | | | | |
| MS-1 | 3.86 × 10⁻⁸ | 6.35 × 10⁻⁹ | 9.59 × 10⁻⁹ | 4.43 × 10⁻⁹ | 6.07 (±0.11) | 2.15 (±0.07) |
| | (±2.3 × 10⁻⁹) | (±4.9 × 10⁻¹⁰) | | | | |
| MS-2 | 8.45 × 10⁻⁹ | 2.06 × 10⁻⁹ | 1.06 × 10⁻⁸ | 4.56 × 10⁻⁹ | 4.10 (±0.07) | 2.33 (±0.01) |
| | (±3.8 × 10⁻¹⁰) | (±5.4 × 10⁻¹⁰) | | | | |

^{a} These values are expressed as mean values and the ± RSD values are based on three measurements.
^{b}In EIS experiments, Zn²⁺ values were measured in the presence of TU and Cu²⁺ values were measured in the presence of PP.

Peptides are valuable candidates for biosensing. Their ability to easily change conformation upon interaction with their natural binders can be translated to electrical sensing. The conformational changes of OT upon Zn²⁺ and Cu²⁺ binding leads to different monolayer packing motifs and are evident from the AFM and EIS studies. The study leading to the development of the present technology demonstrated that the metal ions-dependent change in the conformation of OT produces a unique electrochemical signal pattern that is the outcome of the collective peptides response on the surface. It has been shown that using this principle produces a very sensitive and selective metal ion biosensor. The OT-Sensor proposed opens new avenues for the development of point-of-care sensing devices for neurodegenerative diseases such as MS that relies on neuropeptides as recognition layer.

1. A sensor unit comprising a substrate functionalized with a plurality of metal binding peptides, each of the plurality of metal-binding peptides being associated with or immobilized onto a surface region of the substrate via one or more modes of association/immobilization selected from (a) indirectly via a linker moiety covalently associated to the metal-binding peptide; (b) directly via one or more atoms or groups native to the metal-binding peptide; and (c) by intercalation into a surface-associated monolayer via an aliphatic group covalently associated to the metal-binding peptide.

2-4. (canceled)

5. A metal binding peptide-based sensor unit for detecting the presence and/or determining the amount of at least one metal ion in an aqueous medium, the sensor unit being according to claim 1.

6-8. (canceled)

9. The sensor unit according to claim 1, wherein the metal-binding peptide is selected from oxytocin (OT), somatostatin, vasopressin and derivatives of any of the aforementioned.

10. The sensor unit according to claim 9, wherein the metal-binding peptide is somatostatin, vasopressin or oxytocin, or a derivative thereof.

11-12. (canceled)

13. The sensor unit according to claim 1, wherein the metal binding peptide is of the general formula I: 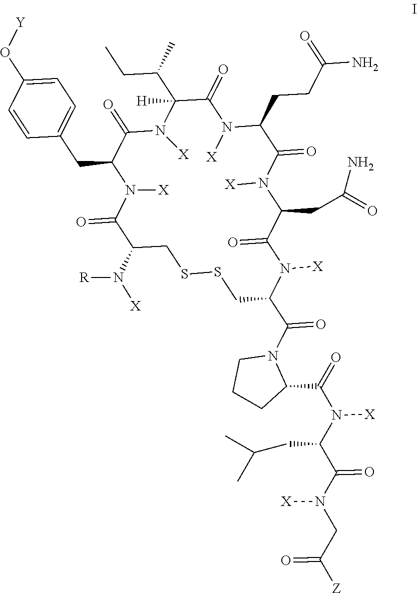
wherein
X is H or a C₁-C₁₆ alkyl;
R is H or a functional group permitting association to the surface or to a bifunctional moiety;
Y is selected from H, PO₃⁻², SO₃⁻¹ and glycan.

14-26. (canceled)

27. The sensor unit according to claim 1, wherein the metal-binding peptide is oxytocin directly associated with the substrate surface via a dissociated disulfide bond.

28. The sensor unit according to claim 1, wherein the metal-binding peptide is oxytocin associated with a substrate surface via a bifunctional group.

29. The sensor unit according to claim 1, wherein the metal-binding peptide is oxytocin associated with a substrate surface via a mercaptoalkanoate group.

30. The sensor unit according to claim 29, wherein the mercaptoalkanoate group comprises between 5 and 15 carbon atoms.

31. The sensor unit according to claim 1, wherein the metal-binding peptide is oxytocin functionalized with at least one aliphatic group, wherein the at least one aliphatic group comprising between 5 and 15 carbon atoms.

32. The sensor unit according to claim 31, wherein the at least one aliphatic group intercalates in a monolayer of aliphatic molecules present on the substrate surface.

33-37. (canceled)

38. The sensor unit according to claim 1, wherein the metal-binding peptide is 8-N-methyl-oxytocin, for detecting the presence of copper and zinc ions in a sample.

39. The sensor unit according to claim 1, wherein the metal-binding peptide is 9-N-methyl-oxytocin, for detecting the presence of zinc ions in a sample.

40. The sensor unit according to claim 1, wherein the metal-binding peptide is 8,9-N,N′-dimethyl-oxytocin, for detecting the presence of zinc ions in a sample.

41. The sensor unit according to claim 1, wherein the metal-binding peptide is 2-N-methyl-oxytocin, for detecting the presence of copper and zinc ions in a sample.

42. The sensor unit according to claim 1, wherein the metal-binding peptide is 3-N-methyl-oxytocin, for detecting the presence of zinc ions in a sample.

43. The sensor unit according to claim 1, wherein the metal-binding peptide is 2,3-N,N′-dimethyl-oxytocin, for detecting the presence of zinc ions in a sample.

44. A method for fabricating a sensor unit according to claim 1, the method comprising forming on a surface region of a substrate an active monolayer comprising a plurality of metal-binding peptide molecules, said metal-binding peptide molecules being associated directly with the surface region via one or more atoms or groups native to the metal-binding peptide, or indirectly via a linker moiety covalently associated to the metal-binding peptide, or by intercalation into a monolayer of alkyl thiols formed on the surface region.

45-60. (canceled)

61. A method for determining the presence of a target metal ion in a sample, or for quantifying a target metal ion in a sample, the method comprising providing a sensor unit according to claim 1; permitting association of metal ions to the metal binding peptide molecules; and measuring at least one signal indicative of the presence of the metal ions in the sample.

62-64. (canceled)

65. A method of diagnosing existence of at least one disease or disorder or predicting the occurrence of a disease or disorder or determining the prevalence of a disease or disorder in a subject or subject population, the disease or disorder being characterized by a chronic or acute abnormality in zinc and/or copper levels in the subject, the method comprising using a sensor according to claim 1, in a sample obtained from the subject, to determine one or more of zinc level, copper level and/or the ratio between the levels of zinc and copper in the sample; and comparing said zinc level, copper level and/or ratio of levels to a normal level thereof; wherein a deviation from said normal level being indicative of the presence, prevalence or occurrence of the disease or disorder.

66-68. (canceled)

69. A device comprising a sensor unit according to claim 1.
